(19) United States
(12) Patent Application Publication
(10) Pub. No.: US 2015/0173318 A1
(43) Pub. Date: Jun. 25, 2015

(54) NOVEL GENETIC FACTOR CAPABLE OF INCREASING YIELD IN MAIZE AND METHOD THEREOF

(71) Applicant: Y.E. Vigor.Corn, DN Misgav (IL)

(72) Inventor: Yoel EFRON, Tivon (IL)

(21) Appl. No.: 14/638,459

(22) Filed: Mar. 4, 2015

Related U.S. Application Data
(63) Continuation-in-part of application No. PCT/IL2013/050754, filed on Sep. 8, 2013.
(60) Provisional application No. 61/698,652, filed on Sep. 9, 2012.

(51) Int. Cl. A01H 5/10; C12Q 1/68; C12N 15/82; A23K 1/14; A23K 1/00; A01G 9/14; A01H 1/04; A23L 1/10

(52) U.S. Cl. CPC A01H 5/10; A01H 1/04; C12Q 1/6895; A23L 1/10; A23K 1/14; A23K 1/007; A01G 9/14; C12N 15/8279; C12N 15/8271; C12Q 2600/13; C12Q 2600/156; A23V 2002/00

(57) ABSTRACT

A maize plant exhibiting additional leaves above the ear (LAE) architecture. The architecture is controlled by a genetic determinant, which shows a non dominant inheritance. The maize plant is an inbred and hybrid variant with an enhanced yield. Also disclosed are methods for developing and producing the inbred and hybrid maize plant with an enhanced yield.

### BACKGROUND OF THE INVENTION

1) Field of the Invention

This invention relates generally to the production of maize, commonly known in the United States as corn and more specifically concerns the development and production of inbred and hybrid maize with additional leaves above the ear to produce maize plants having enhanced yield.

2) Description of Prior Art

There is a real concern on how to feed the world's growing population on constantly decreasing areas of cultivated land.

The ‘Green Revolution’ in wheat and rice is a classical example of significant and dramatic yield increase achieved through a change in plant architecture. In contrast, the trend line for U.S. maize yield has increased only gradually and continually.

Maize is the most widely grown grain crop in the America. Commercial hybrid maize normally grows with 5-7 leaves above the ear placement (see FIG. 1A). The ear that produces the grains normally grows about one-third the way up the plant. The tassel that produces the pollen is found at the top of the plant. The pollen is carried by the wind to the female silk produced on the ear of nearby plants. Hence, maize is naturally cross-pollinated plant which provides a continuing source of variation in genetic constitution.

The focus of maize developments in the last decade was concentrated on producing transgenic genetically modified species with chemical herbicide Glyphosate (round-up) readiness and pest tolerance (e.g. Bt maize gene). However, it has performed nothing in terms of increasing yield.

The growing concern to environmental affects and food safety, recently highlighted by the opposition to the recently developed transgenic corn species, stresses the importance for achieving the higher yield target in a non-transgenic manner.

Maize yield is largely depends on the availability of metabolites produced by the photosynthetic activity of the leaves during grain filling. The photosynthesis during grain filling progressing at the most rapid rate in the leaves is found in the area above the ear.

A number of patents relate to the application of genetic principles to the improvement of maize plants. For example, U.S. Pat. Nos. 2,753,663; 3,594,152 and 3,710,511; each of which concerns the manipulation of genetic male sterility, in the production of hybrid maize seeds. U.S. Pat. No. 4,368,592 relates to the reduction of height of the maize plant by a genetic dominant semi-dwarf allele. U.S. Pat. No. 4,513,532 issued to the Lfy gene relates to a single, dominant genetic factor capable of altering leaf number and distribution in maize. However this dominant Lfy allele causes to the addition of up to 18 leaves above the ear (LAE), and thus rendering the plant unstable and easily tending to fold and droop.

Accordingly, there is an unmet and a long felt need to provide maize with an increased yield potential and improved lodging resistance.

### SUMMARY OF THE INVENTION

It is an object of the present invention to disclose a maize plant exhibiting additional leaves above the ear (LAE) architecture, wherein said architecture is associated with a co dominant allele ELE1 genetic determinant, co-segregating with a molecular marker having a nucleotide sequence corresponding to the nucleotide sequence selected from the group consisting of SEQ ID NO:1 to SEQ ID NO:9 and any combination thereof.

It is another object of the present invention to disclose a maize plant exhibiting additional leaves above the ear (LAE) architecture, wherein the architecture is controlled by a genetic determinant which shows a non dominant inheritance.

It is another object of the present invention to disclose the maize plant as described above, wherein the genetic determinant has a co-dominant inheritance.

It is another object of the present invention to disclose the maize plant as described above, wherein the plant architecture is characterized by additional two or three or four leaves above the ear.

It is another object of the present invention to disclose the maize plant as described above, wherein the plant architecture is characterized by up to four additional leaves above the ear.

It is another object of the present invention to disclose the maize plant as described above, wherein the plant architecture is characterized by less than five additional leaves above the ear.

It is another object of the present invention to disclose the maize plant as described above, wherein the genome of the plant comprises a homozygous configuration of the genetic determinant.

It is another object of the present invention to disclose the maize plant as described above, wherein the genome of the plant comprises a heterozygous configuration of the genetic determinant.

It is another object of the present invention to disclose the maize plant as described above, wherein the additional leaves above the ear architecture is associated with the co dominant allele ELE1 genetic determinant.

It is another object of the present invention to disclose the maize plant as described above, wherein the ELE1 genetic determinant allele is independently segregated from Lfy genetic factor.

It is another object of the present invention to disclose the maize plant as described above, wherein the plant is heterozygous for the ELE1 genetic determinant allele.

It is another object of the present invention to disclose the maize plant as described above, wherein the plant is homozygous for the ELE1 genetic determinant allele.

It is another object of the present invention to disclose the maize plant as described above, wherein the maize plant is characterized by additional leaves above the ear (LAE) architecture in homozygous ELE1/ELE1 genotype configuration as compared with a homozygous ELE2/ELE2 genotype configuration of a normal maize plant.

It is another object of the present invention to disclose the maize plant as described above, wherein the maize plant is characterized by additional leaves above the ear (LAE) architecture in heterozygous ELE1/ELE2 genotype configuration as compared with a homozygous ELE2/ELE2 genotype of normal maize plant.

It is another object of the present invention to disclose the maize plant as described above, wherein the genetic determinant is capable of increasing maize yield as compared to a normal maize plant.

It is another object of the present invention to disclose the maize plant as described above, wherein the plant is capable of producing an increased yield as compared to the yield of a normal maize plant of similar genetic constitution and lacking the genetic determinant ELE1 allele.

It is another object of the present invention to disclose the maize plant as described above, wherein the plant is characterized by an increased yield of up to 40 percent as compared to the yield of a normal maize plant of similar genetic constitution and lacking the genetic determinant ELE1 allele.

It is another object of the present invention to disclose the maize plant as described above, wherein the plant is characterized by an increased yield of about 30 percent as compared to the yield of a normal maize plant of similar genetic constitution and lacking the genetic determinant ELE1 allele.

It is another object of the present invention to disclose the maize plant as described above, wherein the plant is characterized by an increased yield of more than about 30 percent as compared to the yield of a normal maize plant of similar genetic constitution and lacking the genetic determinant ELE1.

It is another object of the present invention to disclose the maize plant as described above, wherein the genetic determinant is capable of conferring an increased plant height without significant effect on ear height.

It is another object of the present invention to disclose the maize plant as described above, wherein the genetic determinant is capable of conferring both an increased average kernel weight and an increased number of kernels per ear at normal planting density.

It is another object of the present invention to disclose the maize plant as described above, wherein the plant has an increased average kernel weight at a normal planting density, as compared to the average kernel weight of a normal maize plant.

It is another object of the present invention to disclose the maize plant as described above, wherein the plant has an increased number of kernels per ear at a normal planting density, as compared to the number of kernels per ear of a normal maize plant.

It is another object of the present invention to disclose the maize plant as described above, wherein at a low planting density the plant further exhibits an increased average number of ears per plant as compared to the number of ears per plant of a normal maize plant.

It is another object of the present invention to disclose the maize plant as described above, wherein the genetic determinant is capable of improving stress tolerance, particularly the genetic determinant is capable of improving at least one characteristic selected from a group comprising: nicking between pollen shed and silk emergence, “Stay Green” characteristic (stem lodging resistance), compensation for low stand by improved prolificacy, parasite tolerance, pests tolerance, draught tolerance, earliness, adaptation to higher density planting, increased dry weight yield and any combination thereof.

It is another object of the present invention to disclose the maize plant as described above, wherein the pant exhibits an improved stress tolerance, particularly an improved characteristic selected from a group comprising: nicking between pollen shed and silk emergence, “Stay Green” characteristic (stem lodging resistance), compensation for low stand by improved prolificacy, parasite tolerance, pests tolerance, draught tolerance, earliness, adaptation to higher density planting, and any combination thereof, as compared to a normal maize plant.

It is another object of the present invention to disclose the maize plant as described above, wherein the genetic determinant confers an increased tolerance to striga.

It is another object of the present invention to disclose the maize plant as described above, wherein the plant has an increased tolerance to striga as compared to the yield of a normal maize plant.

It is another object of the present invention to disclose the maize plant as described above, wherein the tolerance to striga is calculated as the ratio of yield under striga infested and non infested conditions (striga tolerance Index).

It is another object of the present invention to disclose the maize plant as described above, wherein the plant contains a genetic determinant conferring a phenotype with additional leaves above the ear (LAE), which genetic determinant is obtainable from maize line VIGOR B, deposited with NCIMB under accession number 42074.

It is another object of the present invention to disclose the maize plant as described in any of the above, wherein said plant contains a genetic determinant conferring a phenotype with additional leaves above the ear (LAE), which genetic determinant is co-segregating with a molecular marker having a nucleotide sequence corresponding to the nucleotide sequence selected from the group consisting of SEQ ID NO:1 to SEQ ID NO:9 and any combination thereof and is obtainable from maize line VIGOR B, deposited with NCIMB under accession number 42074.

It is another object of the present invention to disclose the maize plant as described above, which can be obtained from a donor plant comprising a genetic determinant conferring a phenotype with additional leaves above the ear (LAE), particularly maize line VIGOR B, deposited with NCIMB under accession number 42074, through introgression of the genetic determinant into a recipient plant with a normal LAE architecture.

It is another object of the present invention to disclose a maize plant line VIGOR B, seeds of which is deposited with NCIMB under accession number 42074, particularly a hybrid maize plant exhibiting additional leaves above the ear (LAE) architecture, produced by using maize plant line VIGOR B as a parent.

It is another object of the present invention to disclose seeds of a plant as described above.

It is another object of the present invention to disclose plant material obtainable from a plant as described above.

It is another object of the present invention to disclose plant parts of a plant as described above.

It is another object of the present invention to disclose maize kernels of a plant as described above.

It is another object of the present invention to disclose the maize kernels which are processed kernels.

It is another object of the present invention to disclose the maize plant as described above, wherein the plant is an inbred, a dihaploid or a hybrid.

It is another object of the present invention to disclose pollen of the maize plant as described above.

It is another object of the present invention to disclose an ovule of the plant as described above.

It is another object of the present invention to disclose the maize plant as described above further comprising an additional trait selected from at least one type of disease resistance and at least one type of stress resistance.

It is another object of the present invention to disclose the maize plant as described above further comprising an additional trait introduced by genetic transformation.

It is another object of the present invention to disclose the maize plant or part thereof, wherein the plant or parts thereof have been transformed so that its genomic material contains one or more transgenes operably linked to one or more regulatory elements.

It is another object of the present invention to disclose a tissue culture of regenerable cells of a maize plant as described above.

It is another object of the present invention to disclose the tissue culture, comprising cells or protoplasts from a tissue selected from the group consisting of leaves, pollen, embryos, roots, root tips, anthers, flowers, fruit and seeds.

It is another object of the present invention to disclose the tissue culture of regenerable cells, wherein the tissue regenerates plants exhibiting an additional leaves above the ear (LAE) architecture, the architecture is controlled by a genetic determinant which shows a co-dominant inheritance.

It is another object of the present invention to disclose a maize plant regenerated from the tissue culture as described above, wherein the plant exhibit an additional leaves above the ear (LAE) architecture, the architecture is controlled by a genetic determinant which shows a co-dominant inheritance.

It is another object of the present invention to disclose a hybrid maize plant comprising a co-dominant genetic allele ELE1, wherein the allele is capable of conferring a phenotype of additional leaves above the ear (LAE).

It is another object of the present invention to disclose a hybrid maize plant characterized by a genetic determinant conferring additional leaves above the ear phenotype, the genetic determinant being capable of transmission to progeny plants substantially as a single non-dominant gene.

It is another object of the present invention to disclose a maize seed derived from the hybrid maize plant as described above.

It is another object of the present invention to disclose the maize seed as described above, wherein the maize plant is a female parent plant.

It is another object of the present invention to disclose the maize seed as described above, wherein the maize plant is a male parent plant.

It is another object of the present invention to disclose the maize plant as described above, wherein the plant exhibits an improved and more efficient root system as compared to the root system of a normal maize plant.

It is another object of the present invention to disclose a method for producing seed maize with additional leaves above the ear (LAE) architecture, the method comprising the steps of: pollinating a first maize plant with pollen of a second maize plant, wherein at least one of the maize plants possess a non dominant genetic determinant controlling additional LAE architecture, and harvesting seeds produced by the pollinated maize plant.

It is another object of the present invention to disclose the method as described above, wherein at least one of the first or second maize plants possess one or two ELE1, co-dominant alleles conferring a phenotype with additional LAE as compared to a normal maize plant of similar genetic constitution and lacking the ELE1 genetic determinant allele.

It is another object of the present invention to disclose the method as described above, wherein the pollen is derived from the first maize plant.

It is another object of the present invention to disclose the method as described above, wherein the pollen is derived from other than the first maize plant.

It is another object of the present invention to disclose a method of producing a maize plant exhibiting an additional leaves above the ear (LAE) architecture, comprising the steps of introgressing from a donor plant, a non dominant ELE1 genetic determinant allele controlling additional LAE architecture, into a recipient plant, with a normal LAE architecture by (a) crossing the donor plant with the recipient plant to obtain a progeny plant exhibiting the additional LAE architecture, (b) screening for and selecting from the progeny plants at least one plant exhibiting an additional leaves above the ear architecture and, optionally, harvesting the resultant progeny seed.

It is another object of the present invention to disclose a method of producing a maize plant exhibiting an additional leaves above the ear (LAE) architecture, comprising the steps of introgressing from a donor plant, a non dominant ELE1 genetic determinant allele, co-segregating with a molecular marker having a nucleotide sequence corresponding to the nucleotide sequence selected from the group consisting of SEQ ID NO:1 to SEQ ID NO:9 and any combination thereof and controlling additional LAE architecture, into a recipient plant by (a) crossing said donor plant with said recipient plant to obtain progeny plants, (b) screening for and selecting from the progeny plants at least one plant exhibiting an additional leaves above the ear architecture co-segregating with a molecular marker having a nucleotide sequence corresponding to the nucleotide sequence selected from the group consisting of SEQ ID NO:1 to SEQ ID NO:9 and any combination thereof, and (c) optionally, harvesting the resultant progeny seed.

It is another object of the present invention to disclose the method, comprising an additional step of screening for and selecting from the progeny plants at least one plant exhibiting an increased yield of up to 40 percent as compared to the yield of a normal maize plant of similar genetic constitution and lacking the ELE1 genetic determinant allele.

It is another object of the present invention to disclose a method of producing a maize plant exhibiting an additional leaves above the ear (LAE) architecture, comprising the steps of introgressing from a donor plant, particularly maize line VIGOR B, deposited with NCIMB under accession number 42074, a non dominant genetic determinant directing or controlling expression of additional leaves above the ear (LAE) into a recipient plant by (a) crossing the donor plant with the recipient plant to obtain a progeny plant exhibiting the additional LAE phenotype, (b) screening for and selecting from the progeny plants at least one plant exhibiting an additional leaves above the ear phenotype co-segregating with a molecular marker having a nucleotide sequence corresponding to the nucleotide sequence selected from the group consisting of SEQ ID NO:1 to SEQ ID NO:9 and any combination thereof, and, optionally, harvesting the resultant progeny seed.

It is another object of the present invention to disclose a method for producing hybrid seed maize exhibiting an additional leaves above the ear (LAE) architecture, the method comprising steps of crossing first and second maize plants, wherein at least one of the maize plants is characterized by the presence of a genetic allele ELE1, conferring additional leaves above the ear (LAE) phenotype, the genetic allele shows co dominant inheritance.

It is another object of the present invention to disclose the method as described above, comprising an additional step of self-pollinating a maize plant possessing the non dominant genetic determinant through at least one generation until the determinant is homozygous, the genetic determinant being capable of conferring a phenotype with additional LAE which is transmittable to progeny as a co-dominant allele and co-segregating with a molecular marker having a nucleotide sequence corresponding to the nucleotide sequence selected from the group consisting of SEQ ID NO:1 to SEQ ID NO:9 and any combination thereof.

It is another object of the present invention to disclose the method as described above, comprising an additional step of backcrossing a maize plant used as a recurrent parent with a second maize plant possessing the genetic determinant being capable of conferring a phenotype with additional leaves above the ear which is transmissible to progeny as co-dominant allele.

It is another object of the present invention to disclose the method as described above, obtained by other means then conventional crossing, particularly different types of genetic engineering methods or any other technique.

It is another object of the present invention to disclose seed maize produced by the method as described above.

It is another object of the present invention to disclose a maize plant produced by the method as described above.

It is another object of the present invention to disclose hybrid maize seed, wherein the seed is characterized by a co-dominant ELE1 genetic allele conferring a phenotype with additional LAE in the maize plant grown from the hybrid maize seed.

It is another object of the present invention to disclose the maize seed, wherein the maize seed is homozygous for the ELE1 genetic allele.

It is another object of the present invention to disclose the maize seed, wherein the maize seed is heterozygous for the ELE1 genetic allele.

It is another object of the present invention to disclose a method for producing inbred maize seed characterized by the presence of a co-dominant ELE1 genetic allele, the method comprising inbreeding a maize plant which is characterized by the genetic allele until the genetic composition of the progeny of such inbreeding becomes substantially stable.

It is another object of the present invention to disclose the method as described above comprising additional steps of: (a) crossing a first maize plant that is the hybrid plant as described above with a second maize plant used as a recurrent parent to yield first progeny seeds; (b) growing the first progeny seed under suitable plant growth conditions to yield an F1 maize plant of the first hybrid plant, the F1 maize plant comprises the co-dominant ELE1 genetic allele conferring additional leaves above the ear; and optionally, (c) crossing the plant obtained in step (b) with itself or with a third maize plant to yield second progeny seeds derived from the first hybrid plant; (d) growing the second progeny seed under suitable plant growth conditions to yield additional maize plant derived of the first hybrid plant, the additional maize plant comprises the co-dominant ELE1 genetic allele conferring additional leaves above the ear; and further optionally, (e) repeating the steps of crossing and growing from a to e or more times to generate further maize plants derived from the first hybrid plant, the further maize plants are characterized by the presence of the co-dominant ELE1 genetic allele conferring additional leaves above the ear.

It is another object of the present invention to disclose maize seed produced by the method as described above.

It is another object of the present invention to disclose a maize plant grown from the seed as described above.

It is another object of the present invention to disclose a genetic determinant being inherited to progeny as a co dominant allele, wherein the genetic determinant is capable of conferring additional leaves above the ear (LAE) architecture.

It is another object of the present invention to disclose a genetic determinant or element having at least 90% sequence identity with the genetic determinant as described above, wherein the genetic determinant or element is capable of conferring additional leaves above the ear (LAE) architecture.

It is another object of the present invention to disclose the genetic determinant, wherein the genetic determinant is being ELE1 co-dominant genetic allele.

It is another object of the present invention to disclose oligonucleotide sequences annealing with the genetic determinant as described above, wherein the sequences are suitable for the detection and production of maize plants having additional leaves above the ear (LAE) architecture.

It is another object of the present invention to disclose a molecular marker having at least 90% nucleotide sequence identity with a nucleotide sequence corresponding to the nucleotide sequence selected from the group consisting of SEQ ID NO:1 to SEQ ID NO:9 and any combination thereof wherein said molecular marker is co-segregating with ELE1 co-dominant genetic determinant capable of conferring additional leaves above the ear (LAE) architecture.

It is another object of the present invention to disclose the molecular marker as defined in any of the above, wherein said molecular marker is further associated with high yield properties selected from the group consisting of: increased plant height without significant effect on ear height, increased average kernel weight, increased number of kernels per ear, increased average number of ears per plant, increased dry weight yield, improved stress tolerance and any combination thereof, as compared to a second maize plant lacking at least one of said molecular markers of said maize plant.

It is another object of the present invention to disclose oligonucleotide sequences annealing with the molecular marker as defined in any of the above, wherein said sequences are suitable for the detection and production of maize plants having additional leaves above the ear (LAE) architecture.

It is another object of the present invention to disclose maize genetic determinants or elements, plants, seeds and plant products as described above for the use in multiple geographical and/or whether-related environments (e.g. tropical, sub-tropic, temperate, etc).

It is another object of the present invention to disclose a method for increasing maize yield production to a commercially relevant extent in multiple geographical and/or whether-related environments or areas comprising growing in the geographical area maize plant as described above.

It is another object of the present invention to disclose a method of producing maize kernels or processed maize kernels as a food product, comprising the steps of: (a) providing a maize plant as described above; (b) propagating the maize plant; (c) allowing the plant to grow corn ears; and, (d) harvesting the kernels of the corn ears.

It is another object of the present invention to disclose use of the seed deposited under accession number NCIMB 42074 for the production of maize kernels.

It is another object of the present invention to disclose use of the maize plant as defined in any of the above or maize kernels grown from the maize plant as described in any of the above as fresh produce, as fresh cut produce, or for processing such as canning and animal feed and silage preparation.

It is another object of the present invention to disclose a maize field or maize greenhouse comprising plants as described above.

### BRIEF DESCRIPTION OF THE FIGURES

In order to better understand the invention and its implementation in practice, a plurality of embodiments will now be described, by way of non-limiting example only, with reference to the accompanying drawings, wherein

FIGS. 1A and 1B present schematic illustration of exemplified physical effects of ELE₁ components compared to normal maize; and

FIG. 2 photographically presents improved stress tolerance of maize plants comprising the ELE1 genetic factor as compared to normal control maize plants.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for providing maize plants exhibiting additional leaves above the ear (LAE) architecture.

The present invention provides a maize plant exhibiting additional leaves above the ear (LAE) architecture, wherein said architecture is associated with a co dominant allele ELE1 genetic determinant, co-segregating with a molecular marker having a nucleotide sequence corresponding to the nucleotide sequence selected from the group consisting of SEQ ID NO:1 to SEQ ID NO:9 and any combination thereof.

According to a further embodiment, the maize plant as defined above exhibits an architecture that co-segregates with a molecular marker profile selected from the group consisting of:
a. at least one molecular marker as set forth in any one of SEQ ID NO:1 to SEQ ID NO:9;
b. at least two molecular markers selected from the group of nucleotide sequences corresponding to the nucleotide sequence as set forth in any one of SEQ ID NO:1 to SEQ ID NO:9;
c. at least three molecular markers selected from the group of nucleotide sequences corresponding to the nucleotide sequence as set forth in any one of SEQ ID NO:1 to SEQ ID NO:9;
d. at least four molecular markers selected from the group of nucleotide sequences corresponding to the nucleotide sequence as set forth in any one of SEQ ID NO:1 to SEQ ID NO:9;
e. at least five molecular markers selected from the group of nucleotide sequences corresponding to the nucleotide sequence as set forth in any one of SEQ ID NO:1 to SEQ ID NO:9;
f. at least six molecular markers selected from the group of nucleotide sequences corresponding to the nucleotide sequence as set forth in any one of SEQ ID NO:1 to SEQ ID NO:9;
g. at least seven molecular markers selected from the group of nucleotide sequences corresponding to the nucleotide sequence as set forth in any one of SEQ ID NO:1 to SEQ ID NO:9;
h. at least eight molecular markers selected from the group of nucleotide sequences corresponding to the nucleotide sequence as set forth in any one of SEQ ID NO:1 to SEQ ID NO:9; and
i. nine molecular markers having a nucleotide sequence corresponding to the nucleotide sequences consisting of SEQ ID NO:1 to SEQ ID NO:9.

It is further within the scope that the plants comprising one of the molecular marker combinations described above exhibit higher yield relative to plants with similar genetic constitution and having either a lower number of molecular markers (SEQ ID NO:1 to SEQ ID NO:9) co-segregating with the additional LAE phenotype or lacking those molecular markers.

According to a further embodiment, it is herein submitted that the yield is positively correlated with the number of markers in the maize plant. In other words, according to certain aspects of the invention, the more molecular markers identified in the maize plant, the more yield it produces.

According to further aspects of the invention, the present invention provides the maize plant as defined in any of the above, wherein the architecture is associated with the ELE QTL located on chromosome 6 between position 163,304,486 and position 169,147,729. This novel ELE QTL encompass the molecular markers selected from the group consisting of SEQ ID NO:1 to SEQ ID NO:9 and any combination thereof.

It is herein defined that the term ‘ELE’ refers to a genetic determinant or factor or QTL or region that is found to affect or to be associated with number of leaves above the ear (LAE).

As used herein the term ‘ELE₁’ refers to an allele which is linked to the unique DNA markers having a nucleotide sequence corresponding to the nucleotide sequence selected from the group consisting of SEQ ID NO:1 to SEQ ID NO:9 and any combination thereof, wherein the markers are co-segregating with additional LAE architecture or phenotype. It is shown by the present invention that the ELE₁ is characterized by a non-dominant inheritance.

According to a further main aspect it is shown by the present disclosure that the additional LAE architecture identified by the unique molecular markers is associated with desirable increased yield characteristics such as increased plant height with or without significant effect on ear height, increased average kernel weight, increased number of kernels per ear, increased average number of ears per plant, improved stress tolerance and any combination thereof, as compared to a second plant with a similar genetic constitution and lacking said ELE1 allele, co-segregating with at least one of said molecular markers of said maize plant.

The term ‘molecular marker profile’ refers hereinafter to a DNA marker set comprising DNA tag sequences representing the uniqueness of a donor line allele, particularly the ELE QTL donor line characterized by the ELE₁ₐₗₗₑₗₑ, co-segregating with the additional LAE phenotype. The DNA marker set comprises at least one molecular marker or DNA tag having a nucleotide sequence corresponding to the nucleotide sequence selected from the group consisting of SEQ ID NO:1 to SEQ ID NO:9 and any combination thereof.

According to specific embodiments, the molecular marker profile of the maize plant with additional LAE architecture of the present invention may be described by the following equation:

N_{M}_{ⱼ}=Σ₍ₙ₎^{M}mᵢ(j), where

M is the total number of molecular markers (i.e. 9 molecular markers);
mᵢ(j) is the presence of molecular marker i for maize plant j; and
N_{M}_{ⱼ} is the number of molecular markers in plant j.

It is further within the scope that the yield rank of maize plant j, characterized by a unique molecular profile, as defined above, relative to a second or reference maize plant may be defined by the following equation:

$$R ( j ) = {\sum }_{i = 1}^{S} ( {N}_{{M}_{j}} > {N}_{{M}_{i}} ) ,$$

where
S is the total number of plants compared to each other; and
R(j) is the yield rank of maize plant j (defined by molecular marker profile N_{M}_{ⱼ}) relative to a second or reference maize plant i (defined by molecular marker profile N_{M}_{ⱼ}).

The present invention provides a non-dominant non-transgenic genetic factor (ELE₁) which is capable of significantly increasing maize yield by adding usually two or three leaves above the ear (LAE). In a specific embodiment, the present invention provides a non-dominant non-transgenic genetic factor (ELE₁) which is capable of significantly increasing maize yield by adding usually two or three leaves above the ear (LAE) in homozygous ELE₁/ELE₁ configuration as compared with a homozygous ELE₂/ELE₂ genotype (normal maize). According to a further embodiment, the heterozygote ELE₁/ELE₂ plant has an intermediate number of LAE.

As used herein, the term ‘about’ refers to a value being ±25% of the defined measure.

The term ‘genetic determinant’ is defined herein as a nucleotide sequence, preferably a DNA sequence that may comprise sequences with various genomic functions such as genes and regulatory elements regions. Genetic determinant may also refer to a nucleotide construct and may be comprised in a vector. Alternatively, a genetic determinant may be transferred from one plant to another by chromosomal recombination after crossing said plants. A genetic determinant may in principle comprise genetic material originating from one or more species. In particular, genetic determinant as used herein refers to a single non dominant gene or multiple genes, a QTL or a haplotype, that determines or controls additional leaves above the ear (LAE) architecture or phenotype in a maize plant. More particularly genetic determinant as used herein refers to ELE non-dominant genetic factor disclosed by the present invention for the first time.

A ‘gene’ is defined herein as a hereditary unit consisting of a sequence of DNA that occupies a specific location on a chromosome and that contains the genetic instruction for a particular characteristic or trait in an organism.

A ‘locus’ is defined herein as the position on a genetic map that a given gene or any other genetic element or factor contributing to a trait occupies on a chromosome of a given species.

As used herein, the term ‘heterozygous’ refers to a genetic configuration existing when different alleles reside at corresponding loci on homologous chromosomes.

As used herein, the term ‘homozygous’ refers to a genetic configuration existing when identical alleles reside at corresponding loci on homologous chromosomes. Homozygosity is defined as absence of segregation after selfing of an individual plant or, if crossed, absence of segregation in F1.

As used herein, the terms ‘hybrid’, ‘hybrid plant’ and ‘hybrid progeny’ refers to an individual produced from genetically different or unlike parents (e.g., parental lines having substantially different genetic constitution). In particular, a hybrid plant refers to a genetically heterozygous or mostly heterozygous individual. More specifically, the phrase F1 hybrid refers to an F1 hybrid produced from a cross between two inbred lines.

As used herein, the phrase ‘inbred line’ refers to a genetically homozygous or nearly homozygous population. An inbred line, for example, can be derived through several cycles of breeding or of selfing. In some embodiments, inbred lines breed true for one or more phenotypic traits of interest. An ‘inbred’, ‘inbred individual’, or ‘inbred progeny’ is an individual sampled from an inbred line.

As used herein, the term ‘trait’ refers to a characteristic or phenotype, e.g., additional leaves above the ear architecture, kernel's yield, stress tolerance and parasite tolerance such as tolerance to striga. A trait may be inherited in a dominant or recessive manner, or in a partial or incomplete-dominant manner. A trait may be monogenic (i.e. determined by a single locus) or polygenic (i.e. determined by more than one locus) or may also result from the mutual interaction among genes or interaction of one or more genes with the environment. A dominant trait results in a complete phenotypic manifestation at heterozygous or homozygous state; a recessive trait manifests itself only when present at homozygous state. According to certain embodiments of present invention, the ELE genetic determinant shows a non dominant inheritance, and more particularly, a co dominant inheritance pattern.

The terms ‘dominant’ and ‘recessive’ refer to the interaction of alleles in producing the phenotype of the heterozygote. Dominance is a genotypic relationship between alleles, as manifested in the phenotype. It is unrelated to the nature of the phenotype itself, e.g., whether it is regarded as normal or abnormal, standard or nonstandard, healthy or diseased, stronger or weaker, or more or less extreme. A dominant trait or allele or dominant genetic factor results in a situation where the phenotype of the heterozygote is completely indistinguishable from that of the dominant homozygote.

The term ‘non dominant’ relates to a genetic configuration inheritance where the phenotype of the heterozygous genotype is an intermediate of the phenotypes of the homozygous genotypes.

The term ‘co-dominant’ or ‘co-dominance’ used herein refers to a genetic configuration inheritance where allelic products co-exist in the phenotype. The term also include incomplete or semi-dominance configuration, where the quantitative interaction of allele products produces an intermediate phenotype. For example the heterozygote ELE₁/ELE₂ plant has an intermediate number of leaves above the ear (LAE) relative to the homozygous ELE₁/ELE₁ or ELE₂/ELE₂ plant.

As used herein, the term ‘allele(s)’ means any of one or more alternative forms or variant forms of various genetic units determinants or factors identical or associated with different forms of a gene or of any kind of identifiable genetic element, all of which alleles relate to at least one trait or characteristic. In a diploid cell, the two alleles of a given gene occupy corresponding loci on a pair of homologous chromosomes and are, therefore, alternative in inheritance. Such alternative or variant forms may be the result of single nucleotide polymorphisms, insertions, inversions, translocations or deletions, or the consequence of gene regulation caused by, for example, by chemical or structural modification, transcription regulation or post-translational modification/regulation. An allele associated with a quantitative trait may comprise alternative or variant forms of various genetic units including those that are identical or associated with a single gene or multiple genes or their products or even a gene disrupting or controlled by a genetic factor contributing to the phenotype represented by said QTL.

As used herein, the term ‘progeny’ refers to the descendant(s) of a particular cross. Typically, progeny result from breeding of two individuals, although some species can be selfed (i.e., the same plant acts as the donor of both male and female gametes). The descendant(s) can be, for example, of the F1, the F2, or any subsequent generation.

As used herein, the terms ‘introgression’, ‘introgressed’ and ‘introgressing’ refer to the process whereby genetic determinants or elements or factors such as genes, a QTL or haplotype of one species, variety or cultivar are transferred into the genome of another species, variety or cultivar, by crossing those species. The crossing may be natural or artificial. The process may optionally be completed by backcrossing to the recurrent parent, in which case introgression refers to infiltration of the genes of one species into the gene pool of another through repeated backcrossing of an interspecific hybrid with one of its parents. An introgression may also be described as a heterologous genetic material stably integrated in the genome of a recipient plant.

The term ‘polymorphism’ is understood within the scope of the invention to refer to the presence in a population of two or more different forms of a gene, genetic marker, or inherited trait or a gene product obtainable, for example, through alternative splicing, DNA methylation, etc.

The term ‘selective breeding’ is understood within the scope of the invention to refer to a program of breeding that uses plants that possess or display desirable traits as parents.

The term ‘second plant’ or ‘reference plant’ or ‘normal maize plant’ or ‘normal plant’ used herein refers to a plant or more specifically to a corn plant i.e. of the species Z. mays having the phenotypic characteristic of about 5 to about 6.6 leaves above the ear, preferably, about 6 leaves above the ear. Such a normal maize plant is genotypically characterized by a homozygous ELE₂/ELE₂ genotype configuration. Thus it is within the scope of the present invention that a normal maize plant lacks the ELE1 genetic factor conferring additional leaves above the ear. It is further within the scope that the genome of such a second or reference plant lacks at least one of the molecular markers of the maize plant of the present invention. Thus it comprises less molecular markers or it is absent of the unique molecular markers, which co-segregate with additional LAE architecture associated with high yield properties phenotype.

In a specific embodiment, a normal maize plant may be used as a recurrent parental line in a breeding scheme. Typically, the plant to be tested is crossed with a ‘normal’ plant of similar genetic constitution and the segregation ratio of the trait in the progeny of the cross is scored. According to main embodiments, phenotypic characteristics such as number of leaves above the ear (LAE), yield, plant height, ear height, ear weight and stress tolerance are compared between the maize plants having the novel ELE1 genetic factor and normal maize plants having similar genetic constitution and lacking the ELE1 genetic determinant.

The terms ‘increased yield’ or ‘high yield’ or ‘ yield components’ used herein refer to genetically enhanced lines or cultivars of crops such as maize that have an increased crop production or increased percentage of usable plant parts, preferably maize kernels or grains. It is within the scope of the invention that the yield produced by a maize plant may include components or parameters such as kernel weight, number of kernels per ear, number of ears per plant, ear weight, ear height, plant height, ear height to plant height ratio, dry weight yield or biomass, relative silking, grain yield, earliness and any combination thereof. According to a main aspect of the invention, the plants provided by the present invention are characterized by increased yield, increased dry weight yield and yield components which is herein surprisingly shown to be correlated with the additional two or three leaves above the ear phenotype conferred by the novel ELE1 genetic determinant.

The term ‘dry weight yield’ refers hereinafter to the plant's measured dry weight or biomass. It is herein acknowledged that since plants have a high composition of water and the level of water in a plant depends on the amount of water in its environment (which is variable and might be difficult to control), using dry weight as a measure of plant growth and yield is important and reliable. In further aspects of the invention, the maize plants comprising the ELE1 genetic determinant conferring additional leaves above the ear can produce an increased yield of up to 40% as compared to the yield of a normal maize plant of similar genetic constitution, lacking said genetic determinant.

In other aspects, the maize plants comprising the ELE1 genetic determinant conferring additional leaves above the ear are possibly characterized by an improved root system, particularly having an extensive and early developed root system, as compared to a normal maize plant, lacking said genetic determinant.

It is within the scope of the present invention that the unique ELE genetic system is possibly correlated with the production of a more efficient and extended root system in the ELE1 maize than in the normal maize lacking the ELE1 factor. Such an extended developed root system brings water and nutrients more easily to the stalk and the leaves and as a result the maize plant barring such a root system is more resistant to stress.

The term ‘nicking’ as used herein generally refers to floral synchronization. More specifically, it refers to the synchronization between pollen shed (anthesis) and silk emergence. Under Favorable conditions, silks emerge 1-3 days after anthesis and remain fertile for about 1 week before senescing. Stress conditions such as water stress, often result in a loss of nick; thus when silks emerged, there is no pollen source, thus barren plants or ears with fewer kernels per ear are produced. Poor ‘nicking’ (lack of synchrony of anthesis and silk emergence) is largely a result of delayed silk emergence. According to some embodiments the maize plants of the present invention, exhibiting additional LAE architecture co-segregating with unique molecular markers exhibit improved yield and tolerance to stress characteristics such as nicking between pollen shed and silk emergence. The term ‘normal plant density’ as used herein refers to maize planting density of about 50,000 plants per hectare (ha). It is herein acknowledged that the optimum density at harvest for a variety is that which yields the most grain when the crop is grown under non-limiting conditions.

The term ‘low plant density’ as used herein refers to maize planting density of about 25,000 plants per hectare.

As used herein, the term ‘population’ means a genetically homogeneous or heterogeneous collection of plants sharing a common genetic derivation.

As used herein, the term ‘variety’ or ‘cultivar’ means a group of similar plants that by structural features and performance can be identified from other varieties within the same species. The term ‘variety’ as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991.

A ‘cultivated maize’ plant is understood within the scope of the invention to refer to a plant that is no longer in the natural state but has been developed by human care and for human use and/or consumption.

As used herein, the term ‘breeding’, and grammatical variants thereof, refer to any process that generates a progeny individual. Breeding can be sexual or asexual, or any combination thereof. Exemplary non-limiting types of breeding include crossing, selfing, doubled haploid derivative generation, and combinations thereof.

‘Backcrossing’ is understood within the scope of the invention to refer to a process in which a hybrid progeny is repeatedly crossed back to one of the parents. Different recurrent parents may be used in subsequent backcrosses.

‘Processed maize plant’ or product is understood within the scope of the invention to refer to maize kernels that are processed into for example (1) canning, and/or (2) concentrated products such as puree, sauce and paste or any other grain-based foods or beverages or processed corn/maize product. Furthermore, the maize plants of the present invention may be used for preparation of animal feed and/or silage.

The term ‘sequence identity’ or ‘sequence homology’ used herein refers to corresponding two or more nucleic acid or protein sequences, that are the same or have a specified percentage of amino acid residues or nucleotides that are the same, when compared and aligned for maximum correspondence, as measured using one of the available sequence comparison algorithms or by visual inspection. If two sequences, which are to be compared with each other, differ in length, sequence identity preferably relates to the percentage of the nucleotide residues of the shorter sequence, which are identical with the nucleotide residues of the longer sequence. As used herein, the percent of identity or homology between two sequences is a function of the number of identical positions shared by the sequences, taking into account the number of gaps, and the length of each gap, which needs to be introduced for optimal alignment of the two sequences. The comparison of sequences and determination of identity percent between two sequences can be accomplished using a mathematical algorithm as known in the relevant art. According to further aspects of the invention, the aforementioned terms refer to variants, homologues and fragments of the indicated nucleotide sequence which possess or perform the same biological function or correlates with the same phenotypic characteristic of the indicated nucleotide sequence.

In other embodiments of the invention, such substantially identical sequences refer to polynucleotide or amino acid sequences that share at least about 80% similarity, preferably at least about 90% similarity, alternatively, about 95%, 96%, 97%, 98% or 99% similarity to the indicated polynucleotide or amino acid sequences.

The term ‘homology’, as used herein, refers to a DNA or amino acid sequence having a degree of sequence similarity in terms of shared amino acid or nucleotide sequences. There may be partial similarity or complete similarity (i.e., identity). For protein sequences, amino acid similarity matrices may be used as are known in different bioinformatics programs (e.g. BLAST, FASTA, Bestfit program—Wisconsin Sequence Analysis Package, Version 8 for Unix, Genetics Computer Group, University Research Park, 575 Science Drive Madison, Wis. 53711, Smith Waterman). Different results may be obtained when performing a particular search with a different matrix. Degrees of similarity for nucleotide sequences are based upon identity matches with penalties made for gaps or insertions required to optimize the alignment, as is well known in the art (e.g. Altschul S. F. et al., 1990, J Mol Biol 215(3):403-10; Altschul S. F. et al., 1997, Nucleic Acids Res. 25:3389-3402). Guidance in determining which amino acid residues may be substituted, inserted, or deleted without abolishing biological or activity may be found using computer programs well known in the art, for example, DNASTAR software.

As used herein, the phrase ‘genetic marker’ or ‘molecular marker’ or ‘DNA marker’ or ‘biomarker’ refers to a feature in an individual's genome e.g., a nucleotide or a polynucleotide sequence that is associated with one or more loci or trait or QTL of interest. In some embodiments, a genetic marker is polymorphic in a population of interest, or the locus occupied by the polymorphism, depending on context. Genetic markers or molecular markers include, for example, single nucleotide polymorphisms (SNPs), DNA tags, indels (i.e., insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome or QTLs that contribute to variability of phenotypic traits. The phrase ‘genetic marker’ or ‘molecular marker’ or ‘biomarker’ can also refer to a polynucleotide sequence complementary or corresponding to a genomic sequence, such as a sequence of a nucleic acid used as a probe or primer.

The present invention provides a set of novel and unique molecular markers co-segregating with additional LAE phenotype, wherein the molecular markers having a nucleotide sequence corresponding to the nucleotide sequence selected from the group consisting of SEQ ID NO:1 to SEQ ID NO:9 and any combination thereof.

A genetic marker can be physically located in a position on a chromosome that is within or outside of the genetic locus with which it is associated (i.e., is intragenic or extragenic, respectively). In some embodiments of the presently disclosed subject matter, the one or more genetic markers comprise a combination of two or more genetic markers. It is also within the scope of the present invention that different combinations of genetic markers are used to identify different traits or phenotypic characteristics as disclosed inter alia.

As used herein, the term ‘co segregating’ is understood within the scope of the invention to refer to the tendency for genes, traits and/or genetic markers to segregate or to be inherited together. Two or more genes, gene alleles or genetic markers that are linked on the same chromosome are transmitted to the same daughter cell leading to the inheritance by the offspring of these genes or alleles together.

More specifically, in the context of the present invention, the term “co-segregation” refers to the fact that the present invention discloses for the first time molecular markers (i.e. at least one molecular marker having a nucleotide sequence corresponding to the nucleotide sequence selected from the group consisting of SEQ ID NO:1 to SEQ ID NO:9) linked to the additional LAE trait that is shown to be associated with high yield in maize. These molecular markers tend to be transmitted together with the allele conferring additional LAE because they are on the same chromosome (reduced recombination between them resulting in a non-random association of their alleles on the same chromosome). “Co-segregation” also refers to the presence of two or more traits, genetic markers or combinations thereof, within a single plant of which at least one is known to be genetic and which cannot be readily explained by chance. In some embodiments, novel genetic markers selected from the group consisting of SEQ ID NO:1 to SEQ ID NO:9 are herein identified to co segregate with the additional leaves above the ear trait.

The term ‘co segregating’ used in the present invention is analogous to coupling or co-inheriting in some of the embodiments of the invention.

The term ‘genetic determinant introgression’ as used herein refers to the incorporation of new genetic determinants or elements such as genes, alleles, QTLs or traits, into a line wherein essentially all of the desired morphological and physiological characteristics of the line are recovered, in addition to the genetically introgressed determinant. Such a process is often used in cultivar development, in which one or a few genetic determinants are transferred to a desired genetic background, preferably by using backcrossing.

The term ‘plant cell culture’ or ‘tissue culture’ as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term ‘plant material’ or ‘plant part’ used herein refers to leaves, stems, roots, root tips, flowers or flower parts, kernels, stalk, cob, grain, ear, pollen, egg cells, zygotes, seeds, seed coat, cuttings, cell or tissue cultures, or any other part or product of a plant or a combination thereof.

It is herein acknowledged that an increased number of leaves above the ear is a desirable trait in maize breeding as it increases leaf area at the period just before flowering until physiological maturity, to enable better adjustment of sink size to source activity and better realization of the potential yield by increasing the proportion of the spiklets filled. In addition, the increased leaf area in the region where photosynthesis is progressing in the most rapid rate, during grain filling, improves metabolites availability to the grains being filled and therefore may increase grain weight.

The present invention provides a novel and unique genetic system (ELE) capable of adding leaves above the ear, preferably 2-3 leaves above the ear in maize. According to a specific embodiment the donor or source of this genetic system may be of tropical background. The ELE genetic system is preferably controlled by a single major gene without dominance, with an additional effect of minor or modifier genes.

A breeding scheme performed in the tropics, with several F2 populations segregating for the number of leaves above the ear has shown an increase in yield and yield components, particularly kernel weight, which was correlated with the increased number of leaves above the ear from about 6 to about 8.

According to a further aspect of the invention, a breeding program was initiated in Israel aimed to transfer the genetic system into maize adapted to temperate climate. Publicly available inbred lines developed in the U.S with particular emphasis on the two well known inbred lines B73 and Mo17 were used as a source of germplasm adapted to the temperate climate.

According to yet another aspect of the invention, the U.S lines were crossed with the ELE genetic material and the F1 plants were advanced to F2. ELE lines were developed by direct self pollination of the F2 plants or by one or two cycles of back crossing followed by self pollination.

General and specific combining ability (GCA and SCA) of the developed lines were tested by test-crosses with normal maize plants i.e. B73 and Mo17 and the newly developed lines were divided to two heterotic groups accordingly.

Experimental hybrids were produced by crossing selected ELE lines from the two heterotic groups or by test crossing the ELE lines with normal maize plants, i.e. B73 and Mo17. The experimental hybrids were tested with and compared with B73×Mo17 hybrids and two commercial recommended hybrids as controls. Several experimental hybrids with significantly higher yields than the controls were surprisingly identified in several trials (see examples below).

According to yet another embodiment of the present invention, more than 1000 hybrid lines comprising the ELE1 genetic factor are produced having improved agricultural characteristics such as increased yiels, lodging resistance, adaptation to higher density of planting, enhanced earliness, improved root system, improved stress tolerance, for example to stress types including parasites, pests and draught, and combinations thereof.

It is thus a core aspect of the invention to clearly demonstrate that yield potential of maize can be significantly increased by adding few leaves above the ear (LAE).

An increased number of LAE controlled by the ELE gene is herein shown to significantly improve yield potential in maize by increasing the photosynthetic active leaf area during the period from just before flowering to physiological maturity. According to certain embodiments of the present invention, both the average kernel weight and the number of kernels per ear are being increased at normal planting density of the plants of the present invention. At a lower planting density, also the average number of ears per plant is increased.

It is herein further disclosed that the addition of LAE can also improve stress tolerance in maize, examples of stress types may include tolerance to parasites such as striga, pests, draught and combinations thereof.

In a further embodiment of the invention, the ELE₁ genetic factor can be transmitted by conventional breeding techniques and by different types of genetic engineering methods, to all types of maize in all possible environments.

Accordingly, the present invention provides a method to increase the number of leaves and leaf area above the ear, in the region where photosynthesis is progressing in the most rapid rate during grain filling. Such a method is further adapted to improve metabolities availability to the grains being filled and consequently to increase yield potential. Hence, one of the objects of this invention is to significantly increase the yield per hectare of maize through a change in plant architecture by adding more leaves above the ear.

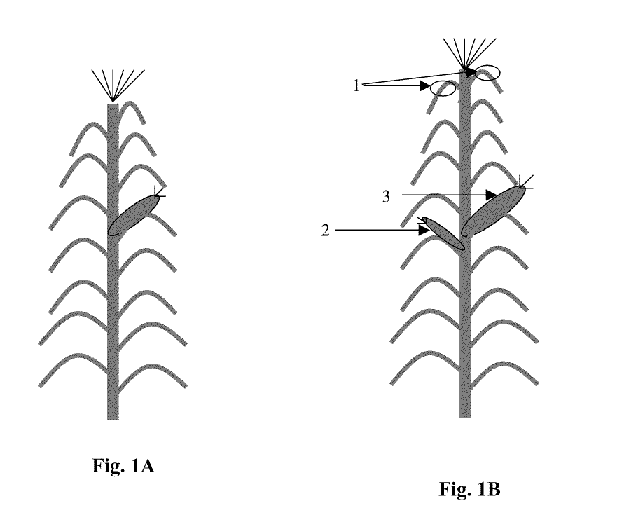

Reference is now made to FIG. 1 schematically presenting exemplified physical effects of ELE1 components as compared to normal maize. FIG. 1A illustrates normal maize preferably characterized by ELE₂/ELE₂ genetic configuration. FIG. 1B illustrates a maize plant comprising the ELE1 genetic determinant, preferably having ELE₁/ELE₁ genetic configuration. As can be seen, the maize plant of FIG. 1B, comprising the ELE1 genetic determinant exhibits additional 2 to 3 leaves above the ear (FIG. 1B, 1) as compared to the normal maize plant (FIG. 1A). Furthermore, the plant with the additional LAE exhibits more cobs (FIG. 1B, 2) and/or larger cobs (FIG. 1B, 3), preferably having more kernels per cob and/or larger kernel weight (FIG. 1B, 2 and 3), as compared to the normal maize plant (FIG. 1A).

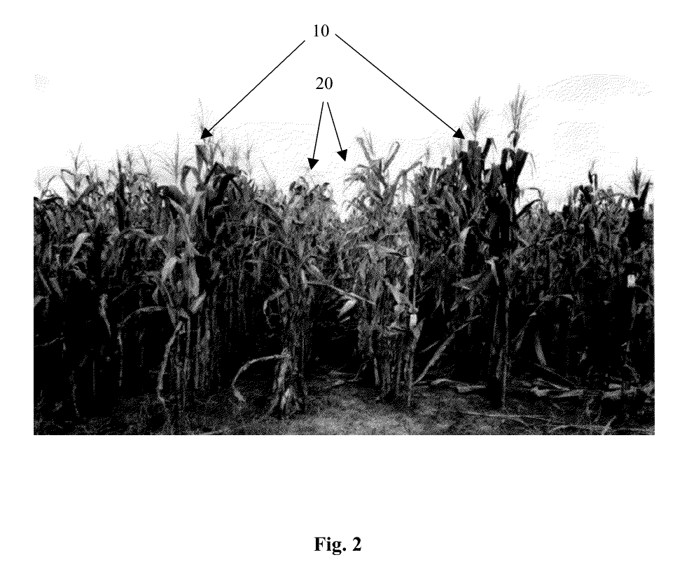

Reference is now made to FIG. 2, presenting a photographic illustration of stress tolerance of maize plants comprising the ELE1 genetic factor as compared to normal control maize plants. As can be seen, the maize plants comprising the ELE1 genetic determinant 10, exhibit improved tolerance to striga hermonthica parasite stress, as compared to normal maize plants 20 lacking the ELE1 factor. In the presence of striga hermonthica infestation, the maize plants comprising the ELE1 genetic determinant 10 are more viable than the normal plants 20; they produce additional leaves and thus are higher than the normal plants 20. These results demonstrate the improved tolerance to parasitic stress such as striga of the maize plants comprising the ELE1 non dominant genetic factor provided by the present invention.

Thus, according to one embodiment, the present invention provides a maize plant exhibiting additional leaves above the ear (LAE) architecture, wherein said architecture is controlled by a genetic determinant which shows a non dominant inheritance.

It is a further embodiment of the present invention to disclose the maize plant as defined above, wherein said genetic determinant has a co-dominant inheritance.

It is a further embodiment of the present invention to disclose the maize plant as defined above, wherein said plant architecture is characterized by additional two or three or four leaves above the ear.

It is a further embodiment of the present invention to disclose the maize plant as defined above, wherein said plant architecture is characterized by up to four additional leaves above the ear.

It is a further embodiment of the present invention to disclose the maize plant as defined above, wherein said plant architecture is characterized by less than five additional leaves above the ear.

It is a further embodiment of the present invention to disclose the maize plant as defined above, wherein the genome of said plant comprises a homozygous configuration of said genetic determinant.

It is a further embodiment of the present invention to disclose the maize plant as defined above, wherein the genome of said plant comprises a heterozygous configuration of said genetic determinant.

It is a further embodiment of the present invention to disclose the maize plant as defined above, wherein the additional leaves above the ear architecture is associated with the co dominant allele ELE1 genetic determinant.

The present invention further provides a method for producing seed maize with additional leaves above the ear (LAE) architecture. The aforementioned method comprising the steps of: pollinating a first maize plant with pollen of a second maize plant, wherein at least one of the maize plants possess a non dominant genetic determinant controlling additional LAE architecture, and harvesting seeds produced by the pollinated maize plant.

More particularly, a cross between maize plant of Tropical origin and a normal maize plant from the U.S Corn Belt origin produced a progeny segregant plant with additional leaves above the ear. The additional leaves above the ear were found to be correlated with a genetic factor designated as ELE₁. The ELE₁ genetic factor act substantially as a non-dominant allele that can add not more than five leaves, preferably two to four leaves and more preferably two to three leaves above the ear (LAE) in homozygous ELE₁/ELE₁ or heterozygous ELE₁/ELE₂ state as compared with a normal ELE₂/ELE₂ homozygous genotype of otherwise similar genetic constitution.

In a further embodiment of the invention, the ELE₁ allele can be transferred between strains of maize by conventional breeding techniques or other (e.g genetic engineering). The resulting derived inbred and/or hybrid strains are characterized by additional leaves above the ear without a noticeable effect on the number of leaves below the ear (see FIG. 1B). As a result, one of the main advantages of the novel genetic system of the present invention is that plant height is being increased, but ear height placement is not affected.

According to a further aspect of the invention, incorporation of the ELE₁ genetic factor into existing hybrids of maize (of multi-environments) is herein shown to improve the yield significantly by up to about 40 percent when compared to the yield of genetically similar normal ELE₂ hybrids.

It is further discloses by the present invention that the ELE₁ allele is not allelic to the Lfy gene described in U.S. Pat. No. 4,513,532 as illustrated in the Examples below. The ELE₁ allele is non-dominant while the Lfy allele is dominant over the lfy of the normal maize. It behaves also differently by being a non-dominant allele compared to the dominant nature of the Lfy gene, its effect is limited to the addition of not more than 5 leaves above the ear and more preferably 2-3 leaves above the ear as compared to the addition of higher number of leaves by the Lfy gene (up to 18 LAE by the Lfy). Similarly its effect on the increased number of days required to reach first silk and pollen (anthesis) is also smaller compared with the effect of the Lfy gene. Furthermore, the ELE₁ allele is superior over the Lfy gene by the ability to confer increased tolerance to stress such as striga infestation and drought, improved lodging resistance, adaptation to higher density planting and earliness as inter alia demonstrated.

It is further within the scope of the present invention that the number of leaves above the ear in an ELE₁/ELE₁ genotype depends on the genetic background of the normal inbred line used at the cross. For example, the number of leaves above the ear may be affected by modifier (minor) genes. Furthermore, the number of leaves above the ear may also depends on the environmental conditions, wherein it is being reduced under stress caused, for example, by Striga hermonthica (a parasitic plant) or it is being increased when the same inbred line is growing under cooler conditions with a longer period from planting to silking.

According to further aspects, the ELE₁ genetic factor of the present invention can be introduced to new elite maize lines by crossing them with the homozygous ELE₁/ELE₁ genotype. The resulting F.sub.1 progeny plants have a phenotype with an intermediate number of leaves above the ear relative to the two parents.

According to one embodiment, the F.sub.1 progeny may be selfed through a number of generations, i.e. 5 generations, or whatever number of generations is necessary or desirable or achieve stable homozygosity.

According to an alternative embodiment, the F.sub.1 plants may be selfed to develop F.sub.2 segregating population. Segregated plants with the higher number of leaves above the ear and other desirable characteristics are being backcrossed with a normal elite ELE₂/ELE₂ line to produce the BC.sub.1 generation.

According to a further embodiment, plants of the BC.sub.1 may be selfed and selected desirable progeny plants are backcrossed again to the same elite normal line. The same procedure may optionally, continue several times, i.e. 5 cycles, which subsequently may convert the known elite inbred to have the ELE₁/ELE₁ genotype.

According to a further embodiment, a combination of both approaches is also possible wherein backcrossing for several generations is followed by several generations of selfing, until homozygosity is being achieved. The advantage of the combined approach is that it takes shorter time to develop ELE₁/ELE₁ inbred lines however the genome of the elite normal lines is not fully recovered.

In a further embodiment, the method of the present invention can be used to add leaves above the ear of all subspecies of maize, specifically including the dent or semi-dent maize, the flint or semi-flint maize, the soft or flower maize, the sweet corns and the pop corns.

In general, the method of the present invention may be used to produce elite hybrid maize (seeds and plants) for commercial maize production. Such hybrid is typically a result of a single cross that is a first generation hybrid between two inbred lines. However, it is also applicable to other types of hybrids such as modified single cross, three-way hybrids, four-way hybrids, top-cross hybrids (a cross between a variety and a line) and varietal cross hybrids. Similarly, open pollinated varieties homozygous for the ELE₁ allele can also be developed by the teachings of the present invention.

According to a further embodiment, the present invention provides two types of hybrids, wherein the ELE gene is either homozygous ELE₁/ELE₁ or heterozygous ELE₁/ELE₂. The first type may be obtained by crossing two homozygous ELE₁/ELE₁ parents. The second type may be obtained by crossing ELE₁/ELE₁ parent with another parent which is ELE₂/ELE₂. The first type may usually have higher number of leaves above the ear than the second type.

According to a further embodiment of the present invention, the incorporation of the ELE₁ genetic factor into existing strains of maize increases the number of leaves above the ear. Consequently it may improve yield by up to 40 percent as compared with the yield of normal maize of similar genetic constitution, but with lower number of leaves above the ear. At normal planting density, i.e. about 50,000 plants per hectare, the yield increase is mainly due to higher kernel's weight and to a lesser extant by higher number of kernels per ear. At a lower density, i.e. about 25,000 plants per hectare, the average number of ears per plant is also increased. This may provide better compensation for low stand of maize plants in the field due to poor germination, as a result of diseases or insect pests attack or due to moisture stress during germination. It is herein further emphesised that the addition of leaves above the ear enables the maize plant to withstand better any stress that reduces the effective photosynthesizing leaf area and thus provides better stress tolerance.

According to a further embodiment, the present invention provides a maize field comprising maize plants as described above.

It is therefore within the scope of the present invention to provide a maize plant exhibiting additional leaves above the ear (LAE) architecture, wherein the architecture is associated with a co dominant allele ELE1 genetic determinant, co-segregating with a molecular marker having a nucleotide sequence corresponding to the nucleotide sequence selected from the group consisting of SEQ ID NO:1 to SEQ ID NO:9 and any combination thereof.

It is further within the scope to disclose the maize plant as described in any of the above, wherein the plant architecture is characterized by not more than five additional leaves above the ear.

It is further within the scope to disclose the maize plant as described in any of the above, wherein the ELE1 genetic determinant allele is independently segregated from Lfy genetic factor.

It is further within the scope to disclose the maize plant as described in any of the above, wherein the plant is heterozygous for the ELE1 genetic determinant allele, or wherein the plant is homozygous for the ELE1 genetic determinant allele.

It is further within the scope to disclose the maize plant as described in any of the above, wherein the plant is characterized by an increased yield of up to about 40 percent as compared to the yield of a normal maize plant lacking the genetic determinant ELE1 allele.

It is further within the scope to disclose the maize plant as described in any of the above, wherein the genetic determinant is capable of conferring an increased average kernel weight, an increased number of kernels per ear, or both, at normal planting density, as compared to a normal maize plant lacking the genetic determinant ELE1 allele.

It is further within the scope to disclose the maize plant as described in any of the above, wherein at a low planting density the plant further exhibits an increased average number of ears per plant as compared to the number of ears per plant of a normal maize plant lacking the genetic determinant ELE1 allele.

It is further within the scope to disclose the maize plant as described in any of the above, wherein the genetic determinant is capable of improving at least one characteristic selected from a group comprising: stress tolerance, dry weight yield, nicking between pollen shed and silk emergence, “Stay Green” characteristic (stem lodging resistance), compensation for low stand by improved prolificacy, parasite tolerance, pests tolerance, draught tolerance, earliness, adaptation to higher density planting, plant height with or without an effect on ear height, tolerance to striga, calculated as the ratio of yield under striga infested and non infested condition, an improved and more developed root system, and any combination thereof, as compared to a normal maize plant lacking the genetic determinant ELE1 allele.

It is further within the scope to disclose the maize plant as described in any of the above, wherein the plant contains a genetic determinant conferring a phenotype with additional leaves above the ear (LAE), which genetic determinant is co-segregating with a molecular marker having a nucleotide sequence corresponding to the nucleotide sequence selected from the group consisting of SEQ ID NO:1 to SEQ ID NO:9 and any combination thereof and is obtainable from maize line VIGOR B, deposited with NCIMB under accession number 42074.

It is further within the scope to disclose the seed, plant material, plant part, maize kernels, processed maize kernels, pollen, and ovule of a plant as defined in any of the above.

It is further within the scope to disclose the maize plant as described in any of the above, wherein the plant is an inbred, a dihaploid or a hybrid.

It is further within the scope to disclose the maize plant as described in any of the above further comprising an additional trait selected from the group consisting of at least one type of disease resistance and at least one type of stress resistance, the trait is introduced by genetic transformation or by introgression.

It is further within the scope to disclose a tissue culture of a maize plant as defined in any of the above, comprising cells, regenerable cells or protoplasts from a tissue selected from the group consisting of leaves, pollen, embryos, roots, root tips, anthers, flowers, fruit and seeds.

It is further within the scope to disclose a method of producing a maize plant exhibiting an additional leaves above the ear (LAE) architecture, comprising the steps of introgressing from a donor plant, a non dominant ELE1 genetic determinant allele, co-segregating with a molecular marker having a nucleotide sequence corresponding to the nucleotide sequence selected from the group consisting of SEQ ID NO:1 to SEQ ID NO:9 and any combination thereof and controlling additional LAE architecture, into a recipient plant by (a) crossing the donor plant with the recipient plant to obtain progeny plants, (b) screening for and selecting from the progeny plants at least one plant exhibiting an additional leaves above the ear architecture co-segregating with a molecular marker having a nucleotide sequence corresponding to the nucleotide sequence selected from the group consisting of SEQ ID NO:1 to SEQ ID NO:9 and any combination thereof, and (c) optionally, harvesting the resultant progeny seed.

It is further within the scope to disclose the method as defined in any of the above, comprising an additional step selected from the group consisting of

(a) screening for and selecting from the progeny plants possessing the molecular marker having a nucleotide sequence corresponding to the nucleotide sequence selected from the group consisting of SEQ ID NO:1 to SEQ ID NO:9 and any combination thereof, at least one plant exhibiting an increased yield of up to 40 percent as compared to the yield of a normal maize plant lacking the ELE1 genetic determinant allele,
(b) crossing a donor plant maize line VIGOR B, deposited with NCIMB under accession number 42074 with a recipient plant to obtain progeny plants, screening for and selecting from the progeny plants at least one plant exhibiting an additional leaves above the ear phenotype co-segregating with a molecular marker having a nucleotide sequence corresponding to the nucleotide sequence selected from the group consisting of SEQ ID NO:1 to SEQ ID NO:9 and any combination thereof, and, optionally, harvesting the resultant progeny seed,
(c) self-pollinating a maize plant possessing the molecular marker through at least one generation until the at least one marker is in a homozygous configuration,
(d) backcrossing a maize plant used as a recurrent parent with a second maize plant possessing the genetic determinant being capable of conferring a phenotype with additional leaves above the ear which is transmissible to progeny as co-dominant allele and is co-segregating with a molecular marker having a nucleotide sequence corresponding to the nucleotide sequence selected from the group consisting of SEQ ID NO:1 to SEQ ID NO:9 and any combination thereof, and (e) the method is obtained by genetic engineering methods.

It is further within the scope of the present invention to disclose a molecular marker having at least 90% nucleotide sequence identity with a nucleotide sequence corresponding to the nucleotide sequence selected from the group consisting of SEQ ID NO:1 to SEQ ID NO:9 and any combination thereof wherein the molecular marker is co-segregating with ELE1 co-dominant genetic determinant capable of conferring additional leaves above the ear (LAE) architecture.

It is further within the scope to disclose oligonucleotide sequences annealing with the molecular marker as defined in any of the above, wherein the sequences are suitable for the detection and production of maize plants having additional leaves above the ear (LAE) architecture.

It is further within the scope to disclose the molecular marker as defined in any of the above, wherein the molecular marker is further associated with high yield properties selected from the group consisting of: increased plant height without significant effect on ear height, increased average kernel weight, increased number of kernels per ear, increased average number of ears per plant, increased dry weight yield, improved stress tolerance and any combination thereof, as compared to a second maize plant lacking at least one of the molecular markers of the maize plant.

It is further within the scope to disclose a method for increasing maize yield production to a commercially relevant extent in multiple geographical and/or weather-related environments or areas comprising growing in the geographical area maize plant as defined in any of the above.

It is further within the scope to disclose a method of producing maize kernels or processed maize kernels as a food product, comprising the steps of:
a. providing a maize plant as defined in any of the above;
b. propagating the maize plant;
c. allowing the plant to grow corn ears; and,
d. harvesting the kernels of the corn ears.

It is further within the scope to disclose use of the maize plant as defined in any of the above or maize kernels grown from a maize plant as defined in any of the above as fresh produce, as fresh cut produce, or for processing such as canning, animal feed and silage preparation.

It is further within the scope to disclose a maize field or maize greenhouse comprising plants as defined in any of the above.

### Deposits:

The following seed samples of Zea mays were deposited on 22 Oct. 2012 with NCIMB, Ferguson Building, Craibstone Estate, Bucksburn, Aberdeen AB21 9YA, Scotland, UK under the provisions of the Budapest Treaty in the name of Y. E. Vigor Corn:

| Accession No. | Date of deposit | Zea mays seed line designation |
|---|---|---|
| NCIMB 42074 | 22 Oct. 2012 | VIGOR B |

The aforementioned genomes can be obtained from said deposited material but can also be obtained from other material. The sequence of the genes obtained from other material may vary from the sequence of the gene in the deposited material (“variant”). Deposit Number NCIMB 42074 or a genetic variant thereof, which refers essentially the same phenotype are available. It is submitted that the seeds deposited under the Budapest treaty and having Deposit Number NCIMB 42074 are a representative example of seeds comprising within its genome at least one molecular marker having a nucleotide sequence corresponding to the nucleotide sequence selected from the group consisting of SEQ ID NO:1 to SEQ ID NO:9 and any combination thereof, which co-segregates with additional LAE phenotype. Seeds of maize plants similar to the above, further comprising at least one additional trait selected from the group consisting of high germination rate, herbicide resistance and insect resistance, are obtainable with regard to a deposit made under the Budapest treaty regulations.

Seed samples were deposited on 22 Oct. 2012 with NCIMB, Ferguson Building, Craibstone Estate, Bucksburn, Aberdeen AB21, 9YA, Scotland, UK under the provisions of the Budapest Treaty. The seed samples include accession number 42074 as designated above.

The following examples are presented in order to more fully illustrate certain embodiments of the invention. They should in no way, however, be construed as limiting the broad scope of the invention. One skilled in the art can readily devise many variations and modifications of the principles disclosed herein without departing from the spirit and scope of the invention.

### EXAMPLES

### Example 1

### The Effect of Planting Season on the Number of Leaves Above the Ear (LAE) in ELE₁/ELE₁ Line

The effect of the planting season was studied in Northern Nigeria which is characterized by two seasons. The normal wet maize planting season that starts in June and a dry cool season that starts in December. It has been shown for the ELE1/ELE1 lines (i.e. lines 11-111 and VIGOR B) that the number of days for silking was higher in the dry season (Table 1). Under these conditions the number of leaves above the ear was increased by about two, i.e. from 8.5 in June planting season to 10.7 in December planting season. This indicates that the number of leaves above the ear is affected by the length of the growing season.

TABLE 1
The effect of planting season on the mean number of days to
silking and the mean number of leaves above the ear (LAE)

| | Planting season | | |
|---|---|---|---|
| | June | July | December |
| No. of days to silk | 53.3 ± 2.1 | 57.1 ± 2.6 | 95.8 ± 4.6 |
| No. of LAE | 8.5 ± 0.94 | 8.9 ± 1.07 | 10.7 ± 1.42 |

### Example 2

### The Effect of Striga Hermonthica Stress on the Average Number of Leaves Above the Ear (LAE)

Striga hermonthica is a parasitic plant causing leaf dryness in maize plants. The effect of stress caused by the parasite was studied in F.sub.2 segregating population of a cross between ELE₁/ELE₁ line (i.e. lines 11-111 and VIGOR B) and normal ELE₂/ELE₂ lines planted at the same time under Striga infested and non-infested field conditions. It was found that as a result of the stress, the average number of LAE was reduced from 7.63 to 6.86 and the frequency distribution was shifted towards the lower number of leaves above the ear (Table 2).

TABLE 2
Frequency distribution (%) of plants with different number of
leaves above the ear in F.sub.2 segregating populations grown under
Striga infested and non-infested field conditions

| Striga | No. | Mean | Plants (%) with the indicated No. of LAE | | | | | | |
|---|---|---|---|---|---|---|---|---|---|
| treatment | Plants | LAE | 4 | 5 | 6 | 7 | 8 | 9 | 10 |
| Infested | 3375 | 6.86 | 0.8 | 10.7 | 29.6 | 36.3 | 17.5 | 5.0 | |
| Non- | 3617 | 7.63 | 0.0 | 1.2 | 12.5 | 36.5 | 27.8 | 16.0 | 5.9 |
| infested | | | | | | | | | |

LAE = Number of leaves above the ear.

### Example 3

### The Effect of ELE1 Genetic Factor on the Number of Leaves Below and Above the Ear

The effect of the ELE₁ genetic factor was studied in progeny hybrids of a cross between ELE₁/ELE₁ line (i.e. lines 11-111 and VIGOR B) and five normal ELE₂/ELE₂ lines. The five heterozygous ELE1/ELE2 hybrids were compared with five hybrids of crosses between the normal ELE₂/ELE₂ lines. It was shown that the mean numbers of leaves below the ear were similar in the two groups, indicating that the ELE₁ genetic factor does not affect the number of leaves below the ear. However it was surprisingly shown that the mean number of leaves above the ear (LAE) was increased in the heterozygous ELE1/ELE2 hybrids relative to the ELE₂/ELE₂ normal hybrid lines i.e. from 6.4 to 7.6 (see Table 3). This result indicates that the ELE₁ genetic factor is capable of increasing the number of leaves above the ear.

TABLE 3
Average number of leaves below and above the ear in five heterozygous
ELE₁/ELE₂ hybrids and five homozygous ELE₂/ELE₂ maize hybrids

| ELE1/ELE2 | No. Leaves | | ELE₂/ELE₂ | No. Leaves | |
|---|---|---|---|---|---|
| Hybrid | BE | AE | Hybrid | BE | AE |
| ELE₁/ELE₁ × | 9.1 ± 0.65 | 7.1 ± 0.32 | 3.57 × 8.21 | 8.6 ± 0.65 | 5.7 ± 0.21 |
| 3-57 | | | | | |
| ELE₁/ELE₁ × | 9.7 ± 0.35 | 6.7 ± 0.12 | 3.57 × 6-133 | 7.6 ± 0.21 | 6.2 ± 0.16 |
| 8-21 | | | | | |
| ELE₁/ELE₁ × | 8.5 ± 0.16 | 7.9 ± 0.12 | 3.57 × 4-11 | 8.5 ± 0.09 | 6.6 ± 0.16 |
| 6-133 | | | | | |
| ELE₁/ELE₁ × | 8.5 ± 0.81 | 8.1 ± 0.10 | 6-133 × 1-105 | 9.7 ± 0.24 | 6.8 ± 0.16 |
| 4-11 | | | | | |
| ELE₁/ ELE₁ × | 9.1 ± 0.24 | 8.3 ± 0.16 | 6-133 × 4-11 | 9.2 ± 0.46 | 6.9 ± 0.17 |
| 1-105 | | | | | |
| Mean | 9.0 ± 0.36 | 7.6 ± 0.75 | Mean | 8.7 ± 0.48 | 6.4 ± 0.20 |

BE = Below the ear
AE = Above the ear

### Example 4

### The Effect of Modifier (Minor) Genes

ELE₁/ELE₁ line i.e. 11-111 or VIGOR B was crossed with five normal ELE₂/ELE₂ lines having different number of leaves above the ear ranging from 5.0 to 6.6. The results obtained demonstrate that in all progeny lines carrying the ELE₁ allele (i.e. ELE₁/ELE₂ and ELE₁/ELE₁), the number of leaves above the ear was increased by one to three leaves. Furthermore, the average number of leaves above the ear in the F.sub.1, F.sub.2 and the two backcrosses populations was shown to be associated with the number of leaves above the ear of the normal parental lines (Table 4). This indicates that the differences between the normal lines are affected by modifiers (minor) genes.

TABLE 4
Average number of leaves above the ear in progeny of crosses
between ELE₁/ELE₁ line and five normal ELE₂/ELE₂ lines

| | | No. of leaves above the ear | | | | |
|---|---|---|---|---|---|---|
| | | Nor- | | | | BC- |
| | ELE₁/ | mal | | | | ELE₁/ |
| Cross | ELE₁ | line | BC-N | F.sub.1 | F.sub.2 | ELE₁ |
| ELE₁/ELE₁ × 4-11 | 8.9 | 6.6 | 7.2 | 8.1 | 8.2 | 8.5 |
| ELE₁/ELE₁ × 1-105 | 8.9 | 6.4 | 7.5 | 8.3 | 8.0 | 8.8 |
| ELE₁/ELE₁ × 6-133 | 8.9 | 5.6 | 7.1 | 7.9 | 7.6 | 8.6 |
| ELE₁/ELE₁ × 8-21 | 8.9 | 5.5 | 6.1 | 6.7 | 7.3 | 8.0 |
| ELE₁/ELE₁ × 3-57 | 8.9 | 5.0 | 6.7 | 7.1 | 7.2 | 7.8 |
| Mean | 8.9 | 5.8 | 6.9 | 7.6 | 7.6 | 8.3 |

BC-N = backcross of F1 plants to the normal line (4-11, 1-105, 6-133, 8-21 and 3-57)
BC- ELE₁/ELE₁ = backcross of F1 plants to ELE₁/ELE₁ line

### Example 5

### Genetic Inheritance of the ELE1 Factor

A total of 13,475 plants of F.sub.2 populations derived from crosses between ELE1/ELE1 line (i.e. 11-111 or VIGOR B) and various normal ELE2/ELE2 lines were tested for the segregation pattern of the number of leaves above the ear. The null hypothesis used was that the number of leaves is genetically controlled by the ELE gene (as demonstrated above), wherein the ELE1 allele that adds leaves above the ear is not dominant to the ELE2 allele present in the normal lines. However, the number of leaves above the ear can be modified somewhat by modifier genes (see Example 4). Accordingly, the F.sub.2 population is expected to segregate in a 1:2:1 ratio for the ELE1/ELE1:ELE1/ELE2:ELE2/ELE2 genotypes provided adjustment is made for the effect of the modifier genes.

The results of this experiment show that the mean number of leaves of all the F.sub.2 plants was 7.05. A deviation of 0.25 leaves below and above the average was allowed for the effect of the modifier genes. Accordingly, the expected number of leaves above the ear for the heterogzyote ELE1/ELE2 should range between 6.8-7.3. The adjusted results fit well with the expected results of the null hypothesis with a probability of 0.50≦P≦0.75 (Table 5).

Thus it is concluded from the above results that the ELE1 allele that adds leaves above the ear is not dominant to the ELE2 allele presented in the normal lines.

TABLE 5
Segregation of leaves number above the ear and
fitness of the adjusted data to a 1:2:1 ratio

| No. | | No. Plants | | | | |
|---|---|---|---|---|---|---|
| LAE | Genotype | Actual | Adjusted | Total | Expected | χ² |
| 5 | ELE2/ELE2 | 671 | 671 | 3346 | 3369 | 0.16 |
| 6 | ELE2/ELE2 | 3343 | 2675 | | | |
| 7 | ELE1/ELE2 | 5230 | 6789 | 6789 | 6738 | 0.39 |
| 8 | ELE1/ELE1 | 2971 | 2080 | 3340 | 3369 | 0.25 |
| 9 | ELE1/ELE1 | 1260 | 1260 | | | |
| | Total | 13475 | 13475 | | | 0.80 |

LAE = Leaves above the ear

### Example 6

### The ELE Gene is not Allelic to the Lfy Gene

Seeds of homozygous Lfy/Lfy line (U.S. Pat. No. 4,513,532) were obtained from the International Institute of Tropical Agriculture (IITA) in Ibadan, Nigeria. This line was crossed with the ELE₁/ELE₁ line (i.e. 11-111 or VIGOR B) and a normal ELE₂/ELE₂ line 1-105. The F.sub.1 plants were advanced to the F.sub.2 generation by selfing and the number of leaves above the ear was counted in the two segregating populations.

The results obtained showed that the number of leaves above the ear ranged from 5 to 14 in the cross with line 1-105 and from 6 to 15 in the cross with ELE₁/ELE₁ line with approximately similar distribution pattern (Table 6). If the ELE and Lfy genes were allelic, all the plants in the cross with ELE₁/ELE₁ line should have had at least 8 leaves and above. The presence of 17.3 percent of plants with 6 or 7 leaves above the ear (Table 6) clearly indicates that the ELE and the Lfy genes are not allelic. Namely, they are two different genes.

TABLE 6
Frequency distribution (%) of the number of leaves above the ear in F.sub.2 segregating
populations of the crosses Lfy/Lfy × ELE1/ELE1 and Lfy/Lfy × ELE2/ELE2

| | Plants (%) with No. leaves above the ear | | | | | | | | | | |
|---|---|---|---|---|---|---|---|---|---|---|---|
| Cross | 5 | 6 | 7 | 8 | 9 | 10 | 11 | 12 | 13 | 14 | 15 |
| Lfy/Lfy × | 0.0 | 4.8 | 12.5 | 15.0 | 14.2 | 13.6 | 11.4 | 10.7 | 7.3 | 4.7 | 2.2 |
| ELE₁/ELE₁ line | | | | | | | | | | | |
| Lfy/Lfy × | 6.3 | 20.0 | 12.4 | 11.3 | 11.7 | 15.0 | 13.8 | 7.1 | 5.0 | 1.3 | 0.0 |
| 1-105 | | | | | | | | | | | |

### Example 7

### The Effect of Increased Number of Leaves Above the Ear on Plant and Ear Heights and Number of Days from Planting to Pollen Shed and Silking

The effect of increased number of leaves above the ear on height and the number of days from planting to flowering was studied in F.sub.2 populations of a cross between ELE₁/ELE₁ line (i.e. 11-111 line or VIGOR B) with several normal ELE2/ELE2 lines segregating for number of leaves above the ear. It was shown that plant height was increased with the increased number of leaves above the ear, but unexpectedly, the ear height was not affected (Table 7). Accordingly, the ratio of ear height to plant height was reduced with the increased number of leaves above the ear. Both, the number of days to anthesis and silking were slightly increased with the increased number of leaves above the ear.

TABLE 7
The effect of leaf number above the ear on ear and plant height and
days to anthesis and silking in F.sub.2 segregating populations

| | | Plant ht. | Ear ht./ | No. days to | |
|---|---|---|---|---|---|
| No. LAE | Ear ht. (cm) | (cm) | Plant ht. | Anthesis | Silking |
| 5 | 84.5 | 166.6 | 0.51 | 53.5 | 55.0 |
| 6 | 85.9 | 178.9 | 0.48 | 54.6 | 55.9 |
| 7 | 87.3 | 191.7 | 0.46 | 54.8 | 56.6 |
| 8 | 82.6 | 195.0 | 0.42 | 54.6 | 56.3 |
| 9 | 83.3 | 198.3 | 0.42 | 58.8 | 60.9 |

LAE = Leaves above the ear

### Example 8

### The Effect of Number of Leaves Above the Ear on Yield Components

The effect of number of leaves above the ear on yield components was studied in F.sub.2 populations of crosses between ELE₁/ELE₁ line (i.e. 11-111 or VIGOR B) and normal inbred lines. It was shown that the number of leaves above the ear is indicative of the ELE genotype. All other genes, unless closely linked, were randomly segregating among the plants. The test was done in two planting densities, the normal density of about 50,000 plants/ha and at a lower density of about 25,000 plants/ha. Plants with six leave above the ear were used as a reference for normal maize (100%) to avoid the possibility that some of the plants with 5 leaves grew in poor soil conditions. Analysis of the plant populations in the different planting densities revealed that at about 50,000 plants/ha the average ear weight was increased by about 32.3 percent in plants having 9 leaves above the ear as compared with plants with 6 leaves above the ear. The average number of ears per plant was not affected (Table 8). It was further demonstrated by this experiment that this increase was due to both an increase in the number of kernels per ear and the average kernal's weight. At a lower density of about 25,000, the average weight of the first ear was increased by about 20 percent and the average number of ears per plant was increased by about 25.0 percent.

The results described above clearly show that the increased number of leaves above the ear controlled by the ELE1 genetic determinant allele affect is surprisingly associated with the increase of yield and desirable yield components such as the number of kernels per ear and the average kernal's weight.

TABLE 8
The effect of number of leaves above the ear (LAE) on yield
and yield component in F.sub.2 segregating populations

| No. | | | | No. | | 1000 | | No. | |
|---|---|---|---|---|---|---|---|---|---|
| leaves | Plants/ | Av. ear | Index | kernels/ | Index | kernel | Index | ears/ | Index |
| AE | ha | wt (g) | (%) | Ear | (%) | wt (g) | (%) | plant | (%) |
| | 50, 000 | | | | | | | | |
| 5 | | 84.0 | 82.2 | 373.9 | 93.5 | 192.0 | 82.4 | 1.05 | 102.9 |
| 6 | | 102.2 | 100.0 | 399.7 | 100.0 | 232.8 | 100.0 | 1.02 | 100.0 |
| 7 | | 114.6 | 112.1 | 422.2 | 105.6 | 245.2 | 105.3 | 1.03 | 101.0 |
| 8 | | 128.8 | 126.0 | 448.7 | 112.2 | 261.3 | 112.2 | 1.03 | 101.0 |
| 9 | | 135.3 | 132.3 | 469.2 | 117.4 | 282.8 | 121.5 | 1.01 | 99.0 |
| | 25, 000 | | | | | | | | |
| 5 | | 100.6 | 80.7 | 372.7 | 83.8 | 239.8 | 93.3 | 1.08 | 96.4 |
| 6 | | 124.7 | 100.0 | 445.0 | 100.0 | 256.8 | 100.0 | 1.12 | 100.0 |
| 7 | | 141.1 | 113.2 | 469.6 | 105.5 | 277.9 | 108.2 | 1.20 | 108.2 |
| 8 | | 148.4 | 119.2 | 486.3 | 109.3 | 285.4 | 111.1 | 1.31 | 117.0 |
| 9 | | 149.9 | 120.2 | 490.7 | 110.3 | 283.1 | 110.2 | 1.40 | 125.0 |

AE = above the ear

### Example 9

### The Effect of Number of Leaves Above the Ear Under Stress Caused by Striga hermonthica

A similar approach as described in Example 8 herein above was taken to test the effect of additional leaves above the ear under stress caused by Striga hermonthica. The same F.sub.2 populations as described above were planted in striga infested and non-infested fields. It was revealed that the stress caused by striga reduced the average yield per plant (Table 9). However, the relative yield increase of plants having 6 LAE to plants having 9 LAE was higher under the stress caused by striga. As a result, it is herein clearly shown that the striga tolerance index calculated as the ratio of yield under striga infested and non-infested conditions was increased with the increased number of leaves above the ears. This result indicates that the addition of leaves above the ear, herein shown to be associated with the ELE1 genetic allele, significantly improves stress tolerance of maize plants.

TABLE 9
The effect of the number of leaves above the ear (LAE)
on maize yield in F.sub.2 populations planted under
striga infested and non-infested fields

| | Striga non-infested | | | Striga infested | | | |
|---|---|---|---|---|---|---|---|
| No. | | Yield/ | | | Yield/ | | |
| leaves | No. | Plant | Index | No. | Plant | Index | Str. Tol |
| AE | Plants | (g) | % | Plants | (g) | % | Index |
| 5 | 45 | 120.3 | 102.9 | 361 | 82.8 | 89.7 | 0.69 |
| 6 | 452 | 117.4 | 100.0 | 1000 | 92.9 | 100.0 | 0.79 |
| 7 | 1320 | 124.5 | 106.0 | 1226 | 106.2 | 115.1 | 0.85 |
| 8 | 1007 | 138.0 | 117.5 | 591 | 112.1 | 121.4 | 0.81 |
| 9 | 578 | 136.7 | 116.4 | 169 | 124.3 | 134.7 | 0.90 |

AE = Above the ear
Str. Tol. Index = Yield under striga infested divided by the yield under striga non-infested conditions

### Example 10

### Yield of ELE Hybrids

Several publicly available U.S Corn Belt inbred lines were converted to ELE₁/ELE₁ genotype. The conversion was done by direct selfing of selected F.sub.2 progenies of a cross between the inbred lines and the ELE₁/ELE₁ line for five generation or selfing after one or two cycles of backcrossing. The developed lines were first test-crossed with B73 and Mo17, a well known publically available heterotic U.S Corn Belt lines. ELE hybrids were developed based on the results of the test-crosses. The performance of the ELE hybrids was tested in comparison to the hybrid B73×Mo17 and two commercially grown hybrids in Israel (Table 10). The results demonstrated the yield superiority of the ELE hybrids compared with the control hybrids. Significant yield increase was obtained, ranging from about 9 to about 36 percent above the normal hybrid B73×Mo17. It was further shown that the ELE hybrids were similar in maturity to the controls.

TABLE 10
Grain yield (T/ha), number of leaves above the ear (LAE) and relative
silking of 12 experimental ELE hybrids and 3 control hybrids

| | | | Grain | | |
|---|---|---|---|---|---|
| | No. leaves | Relative² | yield¹ | Yield Index³ | |
| Hybrid | AE | silking | (T/ha) | (%) | |
| BY 18 × MH 1 | 8.3 | +3.0 | 16.97 | 136 | A |
| BY 27 × MH 5 | 7.8 | +1.8 | 15.3 | 122 | B |
| BY 6 × MH 8 | 7.8 | +0.3 | 15.12 | 121 | BC |
| BY 18 × MH 12 | 8.0 | +3.0 | 15.05 | 120 | BC |
| BY 27 × MH 19 | 7.4 | +0.8 | 14.88 | 119 | BC |
| BY 27 × MH 12 | 7.6 | +2.5 | 14.84 | 119 | BC |
| BY 18 × MH 6 | 7.5 | +2.8 | 14.81 | 118 | BC |
| BY 22 × MH 6 | 7.4 | +1.0 | 14.41 | 115 | BCD |
| BY 27 × MH 8 | 7.9 | 0.0 | 13.92 | 111 | BCDE |
| BY 22 × MH 8 | 7.8 | 0.0 | 13.74 | 110 | BCDE |
| BY 9 × MH 8 | 7.6 | +0.3 | 13.74 | 110 | BCDE |
| BY 27 × MH 6 | 7.4 | +1.5 | 13.64 | 109 | CDE |
| 9220 (comm. Control) | 6.0 | +0.8 | 13.46 | 107 | CDE |
| 8460 (comm. Control) | 6.0 | +0.8 | 12.93 | 103 | DE |
| B73 × Mo17 | 6.0 | 0.0 | 12.50 | 100 | E |

AE = Above the ear
¹Average of four replications
²Number of days to 50% silking relative to B73 × Mo17
³Yields having the same letter are not significantly different at the 5% level (Newman-Keul's test)

### Example 11

### Genetic Markers

This example describes the genetic analysis performed in order to discover novel genetic markers associated with the extra leaves above the ear (LAE) phenotype. The project was designed to meet that challenges by using NRGENE's GenoMAGIC™ algorithm package. Genotype by sequencing (GBS) data of 100 F2 segregating population derived from a cross between B73 and ELE line was produced and analyzed using GenoMAGIC™ platform. Genotype×phenotype (LAE) association analysis was done and the analysis results are reported herein.

### Objectives:

Mapping the genomic region (QTL) affecting leaves No. above the ear (LAE) trait, in a segregating F2 population, of cross between normal maize line B73 and ELE line, and designing unique DNA markers for the associated QTL. These objectives will provide the following:
Genomic location of the QTL/s associated with the LAE phenotype.
Set of unique DNA tags for each discovered QTL.

### DEFINITIONS

Quantitative trait loci (QTLs) are herein refers to as stretches of DNA containing or linked to the genes that underlie a quantitative trait.

DNA tag is herein defined as a short DNA sequence (up to about 100 bp), such as a sequence surrounding a single base-pair change.

DNA marker set herein refers to a set of DNA tag information that all together represents the uniqueness of the favorable donor line allele vs. other alleles in the evaluated population.

### Material and Methods:

### Plant Materials and Phenotypes

An F2 population containing 100 plants from a cross between B73 and ELE line were used in this exemplary project.

Phenotypic data of leaves No. above the ear (LAE) was recorded for each analyzed plant.

### GBS Analysis

The F2 individuals and the parental line (ELE line) were genotyped by sequencing (GBS) in average coverage of 0.2× and 1×, respectively.

Following the GBS data analysis using NRGENE's GenoMAGIC™ platform, the total tags per population after filtration were 10,644,808, and the total segregating tags after filtration were 125,369.

### Results:

### QTL Analysis

The phenotype data and GBS analysis results were processed and analyzed using NRGENE's GenoMAGIC™ platform for QTL analysis.

One major QTL (QTL1) was found on the end of chromosome 6:

Location: chromosome-6, between 163,304,486 to 169,147,729.

Interval size: 5,843,243 bp.

Peak of QTL (top 90%): chromosome-6, between 167,722,310 and 167,901,681.

Interval size: 179,371 bp.

R2 of QTL1: 0.32

P value: 7.4077e-09

LodScore: 8.38

| | | |
|---|---|---|
| Average homozygote 1: | 7.67 | n = 27 |
| Average heterozygote: | 7.00 | n = 45 |
| Average homozygote 2: | 6.29 | n = 28 |

### DNA Tags

All tag sequences and following statistics are based on the peak region (179,371 bp) extended with 100 Kb from each side (chromosome-6 167,622,310 16, 801,681).

Total of 45 segregating tags (100 bp long) in the QTL region, were found. From those tags, 9 (SEQ ID No: 1-9) were with the sequence of the donor allele (see Table 11).

TABLE 11
Location and sequence of the 9 segregating tags

| | Location | | | |
|---|---|---|---|---|
| | Chromosome | start | end | Sequence (100 bp) |
| 1 | 6 | 167,722,214 | 167,722,314 | CAGGCCGTCAGATCCAGATGAACGCCTGAGATCTGATGGCGAGCTCGGACTGGT |
| | | | | TTGCGAACCGGTTCGCTGCGGCACCGCCCGACTCCACGGCGAAGCT |
| 2 | 6 | 167,722,310 | 167,722,410 | AGCTTCGCCGGAGGCGAGCGCATGGCCACAGCGAGAGTCTGGGGCACTGGGAA |
| | | | | AAGGCTCAGGCGGGATCGGGGCGACACGACGAACTCAGTCGTGGGTA |
| 3 | 6 | 167,732,600 | 167,732,700 | AGCTTTGGCACCTGCATAACTTATAGACTAGAGCAAACTAGTTAGTTCAATAATT |
| | | | | TGTGTTGGGCAATTCAATCACCAAAATCATTTAGGAAAAAGGTGT |
| 4 | 6 | 167,749,885 | 167,749,985 | GCAGCGCCACACGACCGGATCTACGCTCAGCCACTCGCTGCAACTACAACCCGA |
| | | | | CGCTACAAGGCACGTTCCTGCAATCTAGAGCTCAACGCGATAAGCT |
| 5 | 6 | 167,768,942 | 167,769,042 | AGCTTCCCCTGCACGCCGATACTACGCAGATCTCGAATGTCAGGGCACAAGAAG |
| | | | | ATTGCTCGAGCGGCGAACATTAGTAGCTCCAAGTACTGCATCTATT |
| 6 | 6 | 167,769,059 | 167,769,159 | TGTCGGGGCTCAGTACCCTTGAGCATGCCCCCCTTAGCTATAAAAGGGGAGGCA |
| | | | | TGCAACGTTACATTACAGGCTCTGGGAGACTCTGGGCTCTCAAGCT |
| 7 | 6 | 167,769,059 | 167,769,159 | TGTCGGGGCTCAGTACCCTTTGTGCATGCCCCCTTAGCTATAAAAGGGGAGGCAT |
| | | | | GCAACGTTACAATATAGGCTCTAGGAGACTCTGGGCTCTCAAGCT |
| 8 | 6 | 167,769,154 | 167,769,254 | AGCTTCCACAACAATCCAACACACAATGGAGTAGGGTATTACGTTCCGGCGGCT |
| | | | | CAAACCACTCTAAACCCTCGCGTGTTCATGTGCTCGGTGATCGCCT |
| 9 | 6 | 167,953,279 | 167,953,379 | ACCGCGTCCAAGCAACGGCTGCGCTAGAGCTTGCCGAACACAACGACACATGT |
| | | | | GGTCACGTAGTATTGCTTCCAGAGGTCGAACTGGCAGTCGCCAAGCT |

### CONCLUSIONS

A single QTL with major effect on the LAE phenotype was discovered in a relatively small genomic interval containing a limited number of putative genes (annotated to B73). Nine novel DNA-tags having a nucleotide sequence corresponding to the nucleotide sequences as set forth in SEQ ID NO:1 to SEQ ID NO:9 have been revealed, co-segregating with the LAE phenotype of the ELE donor line. These novel molecular markers are useful for identifying high yield in maize associated with extra LAE phenotype.

Thus, it is evident by the results disclosed herein that the present invention provides novel and unique molecular markers co-segregating with extra LAE trait. These genetic biomarkers include DNA tag markers identifying extra LAE phenotype surprisingly associated with high yield properties in maize such as increased grain yield, increased kernel weight, increased ear weight, enhanced plant height, improved relative silking time, and striga tolerance as compared to control normal maize line lacking the genetic markers co-segregating with extra LAE trait associated with the ELE1 QTL. Therefore, the markers of Table 11 are useful in detecting high yield maize plants, which exhibit extra LEA phenotype, and screening out low yield plants.

What is claimed is:

1. A maize plant exhibiting additional leaves above the ear (LAE) architecture, wherein said architecture is associated with a co dominant allele ELE1 genetic determinant, co-segregating with a molecular marker having a nucleotide sequence corresponding to the nucleotide sequence selected from the group consisting of SEQ ID NO:1 to SEQ ID NO:9 and any combination thereof.

2. The maize plant of claim 1, wherein said plant architecture is characterized by not more than five additional leaves above the ear.

3. The maize plant of claim 1, wherein said ELE1 genetic determinant allele is independently segregated from Lfy genetic factor.

4. The maize plant of claim 1, wherein said plant is heterozygous for the ELE1 genetic determinant allele, or wherein said plant is homozygous for the ELE1 genetic determinant allele.

5. The maize plant of claim 1, wherein said plant is characterized by an increased yield of up to about 40 percent as compared to the yield of a normal maize plant lacking said genetic determinant ELE1 allele.

6. The maize plant of claim 1, wherein said genetic determinant is capable of conferring an increased average kernel weight, an increased number of kernels per ear, or both, at normal planting density, as compared to a normal maize plant lacking said genetic determinant ELE1 allele; or wherein at a low planting density said plant exhibits an increased average number of ears per plant as compared to the number of ears per plant of a normal maize plant lacking said genetic determinant ELE1 allele.

7. The maize plant of claim 1, wherein said genetic determinant is capable of improving at least one characteristic selected from a group comprising: stress tolerance, dry weight yield, nicking between pollen shed and silk emergence, “Stay Green” characteristic (stem lodging resistance), compensation for low stand by improved prolificacy, parasite tolerance, pests tolerance, draught tolerance, earliness, adaptation to higher density planting, plant height with or without an effect on ear height, tolerance to striga, calculated as the ratio of yield under striga infested and non infested condition, an improved and more developed root system, and any combination thereof, as compared to a normal maize plant lacking said genetic determinant ELE1 allele.

8. The maize plant of claim 1, wherein said plant contains a genetic determinant conferring a phenotype with additional leaves above the ear (LAE), which genetic determinant is co-segregating with a molecular marker having a nucleotide sequence corresponding to the nucleotide sequence selected from the group consisting of SEQ ID NO:1 to SEQ ID NO:9 and any combination thereof and is obtainable from maize line VIGOR B, deposited with NCIMB under accession number 42074.

9. Seed, plant material, plant part, maize kernels, processed maize kernels, pollen, and ovule of a plant according to claim 1.

10. The maize plant according to claim 1, wherein said plant is an inbred, a dihaploid or a hybrid.

11. The maize plant of claim 1 further comprising an additional trait selected from the group consisting of at least one type of disease resistance and at least one type of stress resistance, said trait is introduced by genetic transformation or by introgression.

12. A tissue culture of a maize plant of claim 1, comprising cells, regenerable cells or protoplasts from a tissue selected from the group consisting of leaves, pollen, embryos, roots, root tips, anthers, flowers, fruit and seeds.

13. A method of producing a maize plant exhibiting an additional leaves above the ear (LAE) architecture, comprising the steps of introgressing from a donor plant, a non dominant ELE1 genetic determinant allele, co-segregating with a molecular marker having a nucleotide sequence corresponding to the nucleotide sequence selected from the group consisting of SEQ ID NO:1 to SEQ ID NO:9 and any combination thereof and controlling additional LAE architecture, into a recipient plant by (a) crossing said donor plant with said recipient plant to obtain progeny plants, (b) screening for and selecting from the progeny plants at least one plant exhibiting an additional leaves above the ear architecture co-segregating with a molecular marker having a nucleotide sequence corresponding to the nucleotide sequence selected from the group consisting of SEQ ID NO:1 to SEQ ID NO:9 and any combination thereof, and (c) optionally, harvesting the resultant progeny seed.

14. The method of claim 13, comprising an additional step selected from the group consisting of
(a) screening for and selecting from the progeny plants possessing the molecular marker having a nucleotide sequence corresponding to the nucleotide sequence selected from the group consisting of SEQ ID NO:1 to SEQ ID NO:9 and any combination thereof, at least one plant exhibiting an increased yield of up to 40 percent as compared to the yield of a normal maize plant lacking said ELE1 genetic determinant allele,
(b) crossing a donor plant maize line VIGOR B, deposited with NCIMB under accession number 42074 with a recipient plant to obtain progeny plants, screening for and selecting from the progeny plants at least one plant exhibiting an additional leaves above the ear phenotype co-segregating with a molecular marker having a nucleotide sequence corresponding to the nucleotide sequence selected from the group consisting of SEQ ID NO:1 to SEQ ID NO:9 and any combination thereof, and, optionally, harvesting the resultant progeny seed,
(c) self-pollinating a maize plant possessing said molecular marker through at least one generation until said at least one marker is in a homozygous configuration,
(d) backcrossing a maize plant used as a recurrent parent with a second maize plant possessing said genetic determinant being capable of conferring a phenotype with additional leaves above the ear which is transmissible to progeny as co-dominant allele and is co-segregating with a molecular marker having a nucleotide sequence corresponding to the nucleotide sequence selected from the group consisting of SEQ ID NO:1 to SEQ ID NO:9 and any combination thereof, and (e) said method is obtained by genetic engineering methods

15. A molecular marker having at least 90% nucleotide sequence identity with a nucleotide sequence corresponding to the nucleotide sequence selected from the group consisting of SEQ ID NO:1 to SEQ ID NO:9 and any combination thereof, wherein said molecular marker is co-segregating with ELE1 co-dominant genetic determinant capable of conferring additional leaves above the ear (LAE) architecture.

16. The molecular marker according to claim 15, wherein said molecular marker is further associated with high yield properties selected from the group consisting of: increased plant height without significant effect on ear height, increased average kernel weight, increased number of kernels per ear, increased average number of ears per plant, increased dry weight yield, improved stress tolerance and any combination thereof, as compared to a second maize plant lacking at least one of said molecular markers of said maize plant.

17. Oligonucleotide sequences annealing with the molecular marker of claim 15, wherein said sequences are suitable for the detection and production of maize plants having additional leaves above the ear (LAE) architecture.

18. A method for increasing maize yield production to a commercially relevant extent in multiple geographical and/or weather-related environments or areas comprising growing in said geographical area maize plant according to claim 1.

19. A method of producing maize kernels or processed maize kernels as a food product, comprising the steps of:
a. providing a maize plant according to claim 1;
b. propagating said maize plant;
c. allowing the plant to grow corn ears; and,
d. harvesting the kernels of said corn ears.

20. Use of the maize plant of claim 1 or maize kernels grown from a maize plant according to claim 1 as fresh produce, as fresh cut produce, or for processing such as canning, animal feed and silage preparation.

21. A maize field or maize greenhouse comprising plants of claim 1.


SEQUENCE LISTING (9 sequences)

SEQ ID NO: 1 - DNA, 100 bp, Zea mays
  variation (1)..(100)
caggccgtcagatccagatgaacgcctgagatctgatggcgagctcggactggtttgcgaaccggttcgctgcggcaccgcccgactccacggcgaagct

SEQ ID NO: 2 - DNA, 100 bp, Zea mays
  variation (1)..(100)
agcttcgccggaggcgagcgcatggccacagcgagagtctggggcactgggaaaaggctcaggcgggatcggggcgacacgacgaactcagtcgtgggta

SEQ ID NO: 3 - DNA, 100 bp, Zea mays
  variation (1)..(100)
agctttggcacctgcataacttatagactagagcaaactagttagttcaataatttgtgttgggcaattcaatcaccaaaatcatttaggaaaaaggtgt

SEQ ID NO: 4 - DNA, 100 bp, Zea mays
  variation (1)..(100)
gcagcgccacacgaccggatctacgctcagccactcgctgcaactacaacccgacgctacaaggcacgttcctgcaatctagagctcaacgcgataagct

SEQ ID NO: 5 - DNA, 100 bp, Zea mays
  variation (1)..(100)
agcttcccctgcacgccgatactacgcagatctcgaatgtcagggcacaagaagattgctcgagcggcgaacattagtagctccaagtactgcatctatt

SEQ ID NO: 6 - DNA, 100 bp, Zea mays
  variation (1)..(100)
tgtcggggctcagtacccttgagcatgccccccttagctataaaaggggaggcatgcaacgttacattacaggctctgggagactctgggctctcaagct

SEQ ID NO: 7 - DNA, 100 bp, Zea mays
  variation (1)..(100)
tgtcggggctcagtaccctttgtgcatgcccccttagctataaaaggggaggcatgcaacgttacaatataggctctaggagactctgggctctcaagct

SEQ ID NO: 8 - DNA, 100 bp, Zea mays
  variation (1)..(100)
agcttccacaacaatccaacacacaatggagtagggtattacgttccggcggctcaaaccactctaaaccctcgcgtgttcatgtgctcggtgatcgcct

SEQ ID NO: 9 - DNA, 100 bp, Zea mays
  variation (1)..(100)
accgcgtccaagcaacggctgcgctagagcttgccgaacacaacgacacatgtggtcacgtagtattgcttccagaggtcgaactggcagtcgccaagct